SEQUENCE LISTING (29 sequences)

SEQ ID NO: 1 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 2 - protein, 2117 aa, synthetic construct
MKRTADGSEFESPKKKRKVDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSSGGSSGSETPGTSESATPESSGGSSGGSSTLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSPSGGSKRTADGSEFEPKKKRKV

SEQ ID NO: 3 - protein, 7 aa, synthetic construct
PKKKRRV

SEQ ID NO: 4 - RNA, 79 nt, Human gammaherpesvirus 8
  source 1..79: mol_type = genomic RNA
tgttttggctgggtttttccttgttcgcaccggacacctccagtgaccagacggcaaggtttttatcccagtgtatatt

SEQ ID NO: 5 - RNA, 76 nt, unidentified
  source 1..76: mol_type = unassigned RNA
cgtttgtgttggtttttatgaccagcttggtacaaaacctgctggtgattttttacccaacaaataataaataaaa

SEQ ID NO: 6 - RNA, 83 nt, Homo sapiens
  source 1..83: mol_type = genomic RNA
tagggtcatgaaggtttttcttttcctgagaaaacaacacgtattgttttctcaggttttgctttttggcctttttctagctt

SEQ ID NO: 7 - RNA, 102 nt, synthetic construct
  source 1..102: mol_type = other RNA
tagggtcatgaaggtttttcttttcctgagaaaacaacacgtattgttttctcaggttttgctttttggcctttttctagcttaaaaaaaaaaaaagcaaaa

SEQ ID NO: 8 - RNA, 171 nt, synthetic construct
  source 1..171: mol_type = other RNA
tagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgact

SEQ ID NO: 9 - RNA, 93 nt, synthetic construct
  source 1..93: mol_type = other RNA
gaaggtttttcttttcctgagaaaacaacacgtattgttttctcaggttttgctttttggcctttttctagcttaaaaaaaaaaaaagcaaaa

SEQ ID NO: 10 - RNA, 74 nt, unidentified
  source 1..74: mol_type = unassigned RNA
gccgccgcaggtgtttcttttactgagtgcagcccatggccgcactcaggttttgcttttcaccttcccatctg

SEQ ID NO: 11 - RNA, 101 nt, synthetic construct
  source 1..101: mol_type = other RNA
gccgccgcaggtgtttcttttactgagtgcagcccatggccgcactcaggttttgcttttcaccttcccatctgtgaaagagtgagcaggaaaaagcaaaa

SEQ ID NO: 12 - RNA, 93 nt, synthetic construct
  source 1..93: mol_type = other RNA
aggtgtttcttttactgagtgcagcccatggccgcactcaggttttgcttttcaccttcccatctgtgaaagagtgagcaggaaaaagcaaaa

SEQ ID NO: 13 - RNA, 82 nt, Oryza sativa
  source 1..82: mol_type = genomic RNA
tgttggctgtactcttttctttgtcatggttttctcaaatatgagtttttacatgacaaagtttttaacgaggcagcatgta

SEQ ID NO: 14 - RNA, 69 nt, Mud crab virus
  source 1..69: mol_type = genomic RNA
gagtgtaactcaacagtttttcctaaccacgcgtcgcgtggcaggttttttaatctgagagttacattc

SEQ ID NO: 15 - RNA, 74 nt, Arabidopsis thaliana
  source 1..74: mol_type = genomic RNA
gtgctgtactctttttcctcactatggttttgtcccgaaagggttttcctagtaaggttttaatgaggcagcat

SEQ ID NO: 16 - RNA, 76 nt, Zea mays
  source 1..76: mol_type = genomic RNA
ggctgtactcttttttcctgtctagggtttctcacaagggtgagttttacctagacaggtttttaacgaggcaacc

SEQ ID NO: 17 - RNA, 200 nt, synthetic construct
  source 1..200: mol_type = other RNA
aaaactattttgaagatggtggtgagctgcaaaatagctggatggatttgaatgattgggatgatacatcattgaactgcactttatataaccaaagcttagcagtttgttagataagagtctatgtatgtctctggttaggatgaagttaattttatgtttttaacatggtatttttgaaggagctaatgaaacactgg

SEQ ID NO: 18 - RNA, 200 nt, synthetic construct
  source 1..200: mol_type = other RNA
attgtttctggaaactgcttgccaagacaacatttattaactgttagaacacttgctttatgtttgtgtgtacatattttccacaaatgttataatttatatagtgtggttgaacaggatgcaatcttttgttgtctaaaggtgctgcagttaaaaaaaaaacaaccttttctttcaatatggcatgtagtggagttttt

SEQ ID NO: 19 - protein, 7 aa, synthetic construct
PKKKRKV

SEQ ID NO: 20 - protein, 16 aa, synthetic construct
KRPAATKKAGQAKKKK

SEQ ID NO: 21 - protein, 20 aa, synthetic construct
GRKKRRQRRRPPQPKKKRKV

SEQ ID NO: 22 - protein, 19 aa, synthetic construct
PLSSIFSRIGDPPKKKRKV

SEQ ID NO: 23 - protein, 24 aa, synthetic construct
GALFLGWLGAAGSTMGAPKKKRKV

SEQ ID NO: 24 - protein, 27 aa, synthetic construct
GALFLGFLGAAGSTMGAWSQPKKKRKV

SEQ ID NO: 25 - protein, 21 aa, synthetic construct
KETWWETWWTEWSQPKKKRKV

SEQ ID NO: 26 - DNA, 23 bp, unidentified
  source 1..23: mol_type = unassigned DNA
ggcccagactgagcacgtgatgg

SEQ ID NO: 27 - DNA, 6354 bp, synthetic construct
  source 1..6354: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttgatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggacggcaggcttctgtcgcctctggatccctgggtttgcagaaatggcagcccccctgtaccctctcaccaaaacggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggttgctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 28 - DNA, 6354 bp, synthetic construct
  source 1..6354: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggaggatctagcggaggatcctctggcagcgagacaccaggaacaagcgagtcagcaacaccagagagcagtggcggcagcagcggcggcagcagcaccctaaatatagaagatgagtatcggctacatgagacctcaaaagagccagatgtttctctagggtccacatggctgtctgattttcctcaggcctgggcggaaaccgggggcatgggactggcagttcgccaagctcctctgatcatacctctgaaagcaacctctacccccgtgtccataaaacaataccccatgtcacaagaagccagactggggatcaagccccacatacagagactgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaattgacctggaccagactcccacagggtttcaaaaacagtcccaccctgtttaatgaggcactgcacagagacctagcagacttccggatccagcacccagacttgatcctgctacagtacgtggatgacttactgctggccgccacttctgagctagactgccaacaaggtactcgggccctgttacaaaccctagggaacctcgggtatcgggcctcggccaagaaagcccaaatttgccagaaacaggtcaagtatctggggtatcttctaaaagagggtcagagatggctgactgaggccagaaaagagactgtgatggggcagcctactccgaagacccctcgacaactaagggagttcctagggaaggcaggcttctgtcgcctcttcatccctgggtttgcagaaatggcagcccccctgtaccctctcaccaaaccggggactctgtttaattggggcccagaccaacaaaaggcctatcaagaaatcaagcaagctcttctaactgccccagccctggggttgccagatttgactaagccctttgaactctttgtcgacgagaagcagggctacgccaaaggtgtcctaacgcaaaaactgggaccttggcgtcggccggtggcctacctgtccaaaaagctagacccagtagcagctgggtggcccccttgcctacggatggtagcagccattgccgtactgacaaaggatgcaggcaagctaaccatgggacagccactagtcattctggccccccatgcagtagaggcactagtcaaacaaccccccgaccgctggctttccaacgcccggatgactcactatcaggccttgcttttggacacggaccgggtccagttcggaccggtggtagccctgaacccggctacgctgctcccactgcctgaggaagggctgcaacacaactgccttgatatcctggccgaagcccacggaacccgacccgacctaacggaccagccgctcccagacgccgaccacacctggtacacggatggaagcagtctcttacaagagggacagcgtaaggcgggagctgcggtgaccaccgagaccgaggtaatctgggctaaagccctgccagccgggacatccgctcagcgggctgaactgatagcactcacccaggccctaaagatggcagaaggtaagaagctaaatgtttatactgatagccgttatgcttttgctactgcccatatccatggagaaatatacagaaggcgtgggtggctcacatcagaaggcaaagagatcaaaaataaagacgagatcttggccctactaaaagccctctttctgcccaaaagacttagcataatccattgtccaggacatcaaaagggacacagcgccgaggctagaggcaaccggatggctgaccaagcggcccgaaaggcagccatcacagagactccagacacctctaccctcctcatagaaaattcatcaccctctggcggctcaaaaagaaccgccgacggcagcgaattcgagcccaagaagaagaggaaagtctaa

SEQ ID NO: 29 - protein, 6 aa, synthetic construct
HHHHHH

(19) United States
(12) Patent Application Publication
(10) Pub. No.: US 2024/0409907 A1
(43) Pub. Date: Dec. 12, 2024

(54) IMPROVED PRIME EDITING SYSTEM EFFICIENCY WITH CIS-ACTING REGULATORY ELEMENTS

(71) Applicant: SIGMA-ALDRICH CO. LLC, St. Louis, MO (US)

(72) Inventor: Qiaohua Kang, Ballwin, MO (US)

(21) Appl. No.: 18/691,560

(22) PCT Filed: Sep. 9, 2022
(86) PCT No.: PCT/US2022/076175

§ 371 Date: Mar. 13, 2024

Related U.S. Application Data
(60) Provisional application No. 63/363,247, filed on Apr. 20, 2022.
(60) Provisional application No. 63/243,423, filed on Sep. 13, 2021.

(51) Int. Cl. C12N 9/22; C12N 9/12; C12N 15/11; C12N 15/86

(52) U.S. Cl. CPC C12N 9/22; C12N 9/1276; C12N 15/111; C12N 15/86; C12N 2310/20; C12Y 207/07049

(57) ABSTRACT

The present invention is a synthetic nucleic acid composition comprising: i) a sequence encoding a CRISPR-Cas protein, ii) a sequence encoding a reverse transcriptase, and iii) a sequence encoding a cis-acting regulatory element, and methods of use thereof.

### CROSS-REFERENCE TO RELATED APPLICATIONS

The present application claims the benefit of priority of US Provisional Application Nos. 63/243,423, filed Sep. 13, 2021 and 63/363,247, filed Apr. 20, 2022, the entire contents of both applications are incorporated herein by reference.

### BACKGROUND

Targeted genome modification is a powerful tool for genetic manipulation of DNA, including the manipulation of eukaryotic cells, embryos and animals. For example, exogenous sequences can be integrated at targeted genomic locations and/or specific endogenous DNA (e.g., chromosomal) sequences can be deleted, inactivated or modified. Previous to the CRISPR/Cas9 methodology (Perez-Pinera P, Ousterout D G, Gersbach C A, Advances in targeted genome editing, Curr Opin Chem Biol., 2012, 16(3-4):268-77; Hsu P D, Lander E S, Zhang F, Development and Applications of CRISPR-Cas9 for Genome Engineering, Cell, 2014, 157(6): 1262-1278), methods relied on the use of engineered nuclease enzymes, such as, for example, zinc finger nucleases (ZFNs) or transcription activator-like effector nucleases (TALENs). These chimeric nucleases contain programmable, sequence-specific DNA-binding modules linked to a nonspecific DNA cleavage domain. Each new genomic target, however, requires the design of a new ZFN or TALEN comprising a novel sequence-specific DNA-binding module. Thus, these custom designed nucleases tend to be costly and time-consuming to prepare. Moreover, the specificities of ZFNs and TALENS are such that they can mediate off-target cleavages.

Crispr/Cas9 technology has greatly enhanced the ability or workers to target and manipulate DNA sequences, especially in vivo eukaryotic sequences. However, CRISPR systems are not without their own limitations. For example, CRISPR/Cas9 systems work by making double stand breaks (DSBs) that allow for the insertion, deletion or base substitution at the break site. However, DSBs are also associated with undesired outcomes including, for example, translocations. Further, know pathological alleles arise from very precise, albeit unfortunate, insertions, deletions or base substitutions that require precise gene editing to correct. Current technology is often lacking in the precision and/or efficiency necessary or results in unacceptable outcomes.

Recently Anzalone, et al., introduced a CRISPR-based system called prime editing. Anzalone, et al., Nature, 5 Dec. 2019. 576:149-157. The prime editing system permits “search and replace” genome editing without double-strand breaks or donor DNA. The system is described by the authors to allow for “genome editing that mediates targeted insertions, deletions and all 12 possible base-to-base conversions, and combinations thereof, in human cells.” Ibid., page 149. However, it is known in the art that with regard to prime editing, “ . . . factors that affect efficiency have not been extensively investigated.” Kim, et al., Nature Biotechnology, February 2021, Vol 39, 198-206. What is needed are compositions and methods to improve the efficiency of the prime editing system.

### SUMMARY OF THE INVENTION

Among the various aspects of the present invention are compositions and methods that substantially increase the efficiency of the prime editing system.

In a non-limiting exemplification, the Prime Editing System (PES) comprises a Cas9(H840A) nickase-reverse transcriptase (RT) fusion protein and a prime editing guide RNA (pegRNA). The PE:pegRNA complex binds the target DNA and nicks the PAM (protospacer adjacent motif)-containing strand. The resulting 3′ end hybridizes to the primer-binding site, and then primes reverse transcription of new DNA containing the desired edit using the transcriptase template of the pegRNA; Equilibration between the edited 3′ flap and the unedited 5′ flap, cellular 5′ flap cleavage and ligation, and DNA repair results in stably edited DNA (Anzalone, et al., 2019) (See, FIG. 1; prior art). Current prime editors as exemplified by the system represented in FIG. 1 do not display desired high efficiencies, which limit their further use either in research or therapeutic fields.

Current prime editing technology employs, for example, the Cas9(H840A) nickase-reverse transcriptase (RT) fusion protein coupled with the prime editing guide RNA (pegRNA). The desired edit by prime editing technology depends on the equilibration between the edited 3′ flap and the unedited 5′ flap. Due to the large size of Cas9(H840A) nickase-RT fusion protein, stable and efficient expression of said Cas9(H840A) nickase-RT fusion protein in target cells is consistently a challenge to achieve and this affects obtaining the desired edits by using the PES.

To address this issue the present inventors have incorporated cis-acting regulatory element(s) (e.g., dENE or sRSM1) in the Cas9(H840A) nickase-RT fusion expression cassette (FIG. 2) to improve its mRNA stability and protein expression believing this would greatly enhance the efficiency of prime editing as well as any other gene editing technology that involves effector protein expression including CRISPR-Cas9, CRISPR-Cas9 nickase, CRISPRi, CRISPRa, etc. As is shown in the exemplification section, below, this invention greatly enhances the efficiencies of current prime editing technology without changing the features of the desired edits and addition of any extra component to the prime editing complex.

Thus, the present invention is directed toward compositions and methods for substantially improving the efficiency of prime editing.

In one aspect, the present invention contemplates a synthetic nucleic acid composition comprising: i) a sequence encoding a CRISPR-Cas protein, ii) a sequence encoding a reverse transcriptase, and iii) a sequence encoding a cis-acting regulatory element.

The CRISPR-Cas protein encoded by the synthetic nucleic acid composition of the present invention may be any CRISPR-Cas protein known to one of ordinary skill in the art. In one aspect of the present invention, the CRISPR-Cas protein is nCas9-H840A.

The reverse transcriptase encoded by the synthetic nucleic acid composition of the may be any reverse transcriptase known to one of ordinary skill in the art. In one aspect of the present invention, the reverse transcriptase is M-MLV-RT.

The cis-acting regulatory element encoded by the synthetic nucleic acid composition of the present invention may be any cis-acting regulatory element known to one of skill in the art. In one aspect of the present invention, cis-acting regulatory element is dENE, ENE or sRSM1.

In one aspect of the present invention the synthetic nucleic acid composition of the present invention is DNA.

In one aspect of the invention, the synthetic nucleic acid composition of the present invention is RNA.

The present invention contemplates that the synthetic nucleic acid composition of the present invention further comprises an expression promotor.

The present invention further contemplates that the synthetic nucleic acid composition of the present invention in an expression vector.

The present invention further contemplates that the synthetic nucleic acid composition of the present invention is incorporated into a transfection virus.

The present invention further contemplates that the cis-acting regulatory element of the synthetic nucleic acid composition of the present invention is located after the stop codon of the CRISPR-Cas9 sequence and before an mRNA terminator.

The present invention further contemplates that the synthetic nucleic acid composition of the present invention further comprises a prime editing guide RNA (pegRNA), wherein said pegRNA is derived from one of PE1, PE2 and PE2.

The present invention further contemplates an amino acid sequence encoded by the synthetic nucleic acid composition of the present invention.

The present invention is also directed toward methods of use. In one aspect, the present invention contemplates a method of modifying an endogenous DNA sequence, the method comprising: providing: i) an operable expression vector comprising a synthetic nucleic acid composition comprising: 1) a sequence encoding a CRISPR-Cas type II system protein, 2) a sequence encoding a reverse transcriptase, and 3) a sequence comprising a cis-acting regulatory element; ii) a prime editing guide RNA (pegRNA) comprising a prime binding site (PBS); and iii) a cell comprising a target endogenous DNA sequence being at least 50% complementary to the PBS; transfecting the cell comprising the endogenous DNA sequence of interest with the synthetic nucleic acid composition and pegRNA of the present invention; and culturing said transfected cell such that the desired modification is made to the endogenous DNA sequence.

The present further contemplates that that the synthetic nucleic acid composition use in the method of the present invention may be any CRISPR-Cas protein known to one of ordinary skill in the art. In one aspect of the present invention, the CRISPR-Cas type II system protein is a Cas9 protein.

It is further contemplated by the method of the present invention that the endogenous DNA sequence is at least 75% complementary to the PBS.

It is further contemplated by the method of the present invention that the endogenous DNA sequence is at least 90% complementary to the PBS.

It is further contemplated by the method of the present invention that the endogenous DNA sequence is at least 95% complementary to the PBS.

It is further contemplated by the method of the present invention that the endogenous DNA sequence is at least 98% complementary to the PBS.

It is further contemplated by the method of the present invention that the endogenous DNA sequence is 100% complementary to the PBS.

It is further contemplated by the method of the present invention that the CRISPR-Cas protein may be any CRISPR-Cas protein known to one of ordinary skill in the art. In one aspect of the present invention it is be nCas9-H840A.

It is further contemplated by the method of the present invention that the reverse transcriptase may be any reverse transcriptase known to one of ordinary skill in the art. In one aspect of the present invention it is M-MLV-RT.

It is further contemplated by the method of the present invention that the cis-acting regulatory element may be any cis-acting regulatory element known to one of skill in the art. In one aspect of the present invention the cis-acting regulatory element is selected from dENE, ENE and sRSM1.

It is further contemplated by the method of the present invention that the operable expression vector encoding the synthetic nucleic acid of the present invention is DNA.

It is further contemplated by the method of the present invention that the operable expression vector encoding the synthetic nucleic acid of the present invention is RNA

It is further contemplated by the method of the present invention that the synthetic nucleic acid composition of the present invention is incorporated into a transfection virus.

It is further contemplated by the method of the present invention that synthetic nucleic acid composition the cis-acting regulatory element of the present invention is located after the stop codon of the CRISPR-Cas9 sequence and before an mRNA terminator.

It is further contemplated by the method of the present invention that the pegRNA is derived from one of PE1, PE2 and PE3.

It is further contemplated by the method of the present invention that the CRISPR/Cas type II system protein is introduced into the cell encoded in an operable expression vector

### BRIEF DESCRIPTION OF THE DRAWINGS

FIG. 1 shows a diagram of prime editing technology as is illustrated in the prior art. nCas9(H840A)=Cas9(H840A nickase; RT=Reverse Transcriptase; PBS=prime binding site.

FIG. 2 shows a diagram of the introduction of a cis-acting regulatory element to the prime editing expression cassette.

FIG. 3 shows PE2-dENE enhances the editing efficiency of PE2.

FIG. 4 shows PE3-dENE enhances the editing efficiency of PE3.

FIG. 5 shows 3′-UTR dENE improves PE editing efficiency on HEK3 target in K562 cells.

FIG. 6 shows 3′-UTR dENE does not improve PE editing efficiency on HEK3 target in HEK293 cells.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention: Singleton, et al., Dictionary of Microbiology and Molecular Biology (2nd ed. 1994); The Cambridge Dictionary of Science and Technology (Walker ed., 1988); The Glossary of Genetics, 5th Ed., R. Rieger, et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

The transitional phrases “comprising,” “consisting essentially of” and “consisting of” have the meanings as given in MPEP 2111.03 (Manual of Patent Examining Procedure; United States Patent and Trademark Office). Any claims using the transitional phrase “consisting essentially of” will be understood as reciting only essential elements of the invention and any other elements recited in dependent claims are understood to be non-essential to the invention recited in the claim from which they depend.

As used herein, the term “endogenous sequence” refers to a chromosomal sequence that is native to the cell.

The term “exogenous,” as used herein, refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “synthetic nucleic acid” refers to a nucleotide sequence synthesized in vitro (for example, in a lab and either manually or with a nucleic acid synthesizer) and in which the sequence is not found in nature. The sequence may be, for example, DNA or RNA or a modification thereof as described below, may be any length and may be any sequence of nucleotides so long as the sequence is not naturally occurring.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website.

As various changes could be made in the above-described cells and methods without departing from the scope of the invention, it is intended that all matter contained in the above description and in the examples given below, shall be interpreted as illustrative and not in a limiting sense.

### Prime Editing System

The Prime Editing System (PES) is an improvement of the CRISPR/Cas9 technology. As first described by Anzalone, et al., the PES uses a prime editing guide RNA (pegRNA) to direct the CRISPR/Cas9 complex to the desired target site in the genome. The PEG is described as (Marzec, et al., Trends in Cell Biology, April 2020, 33:4, 257-259) containing not only the spacer that is complementary to the target DNA strand but also a primer binding site (PBS) region and the sequence that will be introduced into the targeted DNA region. The PBS is complementary to the second DNA strand and will create a primer for the reverse transcriptase (RT) that is linked to the Cas9 nickase. The RT is an RNA-dependent polymerase that uses the sequence from the pegRNA as a template. The sequence is copied directly from the peg DNA into the target DNA sequences, thus, altering the target sequence in the desired manner.

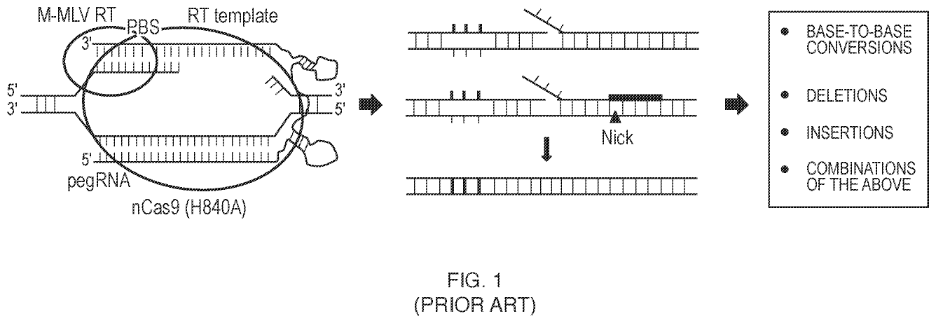

In a non-limiting exemplification, the Prime Editing (PE) comprises a Cas9(H840A) nickase-reverse transcriptase (RT) fusion protein and a prime editing guide RNA (pegRNA); The PE:pegRNA complex binds the target DNA and nicks the PAM-containing strand. The resulting 3′ end hybridizes to the primer-binding site, and then primes reverse transcription of new DNA containing the desired edit using the transcriptase template of the pegRNA; Equilibration between the edited 3′ flap and the unedited 5′ flap, endogenous cellular 5′ flap cleavage and ligation, and DNA repair results in stably edited DNA (Anzalone et al., 2019; ref. FIG. 1). To date, several versions of prime editors (PEs) have been developed. PE1 [SEQ ID NO: 1] is designated by using wild-type Moloney murine leukemia virus reverse transcriptase (M-MLV RT) fused to the C terminus of Cas9(H840A) nickase. PE2 [SEQ ID NO: 2] uses an engineered M-MLV RT. PE3 [SEQ ID NO: 3] is defined by introducing an additional guide RNA to nick the non-edited strand, which increases editing efficiency, albeit indel frequency. In PE3b (Anzalone, et al.), this nicking single guide RNA (sgRNA) targets the edited sequence, thereby preventing nicking of the non-edited strand until after editing occurs, resulting in fewer indels in mammalian cells.

### Cis-Regulatory Elements

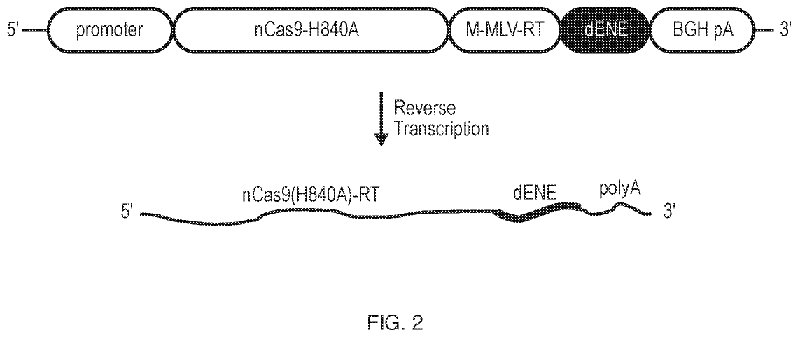

The present invention substantially improves the efficient of the prime editing system by incorporating one or more Cis-regulatory elements (CREs) into the system. (see, FIG. 2) One of ordinary skill in the art will understand, armed with the teachings of this specification, that Cis-regulatory elements other than the ones specifically exemplified herein may also be suitable use with the present invention and that it is within the skill and knowledge of the ordinary practitioner of the art, following the teaching of the present specification, to screen said Cis-regulatory elements without undue experimentation.

As Wittkapp and Kalay teach us (Nature Reviews Genetics, January 2012, vol. 13, pp. 59-69), Cis-regulatory element(s) is a term for a collection of transcription factor binding sites and other non-coding DNA that are sufficient to activate (or inhibit) transcription in a defined spatial and/or temporal expression domain. Cis-regulatory elements are a type of Cis-regulatory sequence needed to activate and sustain transcription. They are composed of DNA (typically, non-coding DNA) containing binding sites for transcription factors and other regulatory molecules. Promotors, enhancers and silencers are the most commonly recognized types of CREs.

Promotors are required for transcription in eukaryotes but, typically, produce only basal levels of mRNA. Enhancers are more variable that promotors and aid in the upregulation of expression and transcription.

Another way of viewing the regulation of gene expression with cis- and trans-regulating elements is that cis-regulatory elements are often binding sites for one or more trans-acting factors. Cis-regulatory elements are usually present on the same molecule of DNA as the gene they regulate whereas trans-regulatory elements can regulate genes distant from the gene from which they were transcribed. Transcription factors are an example of trans-acting factors.

Enhancers are CREs that influence (enhance) the transcription of genes on the same molecule of DNA and can be found upstream, downstream, within the introns, or even relatively far away from the gene they regulate. Multiple enhancers can act in a coordinated fashion to regulate transcription of one gene. (ibid., Wittkapp and Kalay.) A number of genome-wide sequencing projects have revealed that enhancers are often transcribed to long non-coding RNA (lncRNA) or enhancer RNA (eRNA), whose changes in levels frequently correlate with those of the target gene mRNA. (Melamed P., Yosefzun Y., et al., Transcription, 2 Mar. 2016, 7 (1): 26-31.)

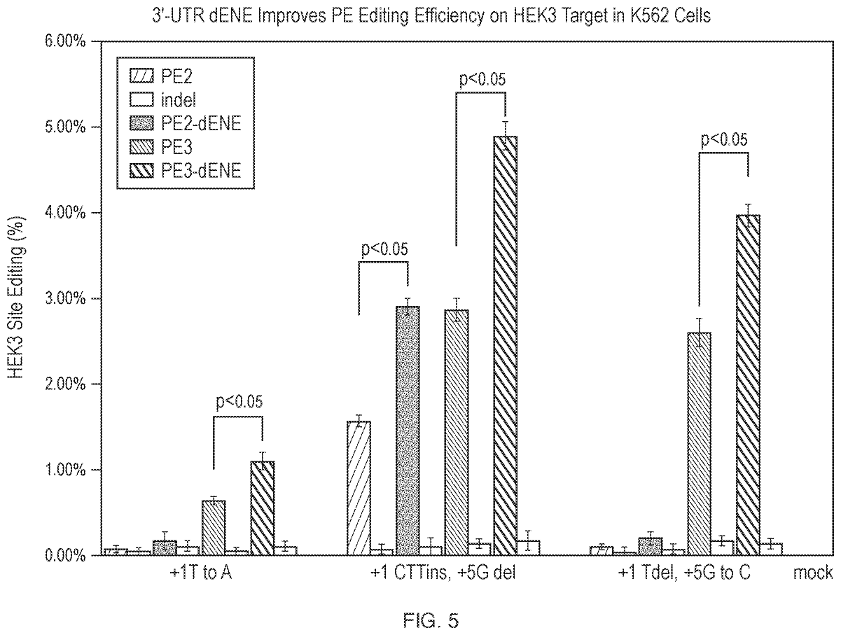

While it is contemplated by the inventors that any cis-acting regulatory element will convey an advantageous benefit to CRISPR-based nucleic acid modification technology, preferred non-limiting examples of suitable cis-acting regulatory elements are:
Element for Nuclear Expression (ENE): containing a U-rich internal loop (URIL) flanked by short double helices that confers RNA stabilization, examples include ENE from Kaposi's sarcoma-associated herpesvirus (KSHV), ENE from human metastasis-associated lung adenocarcinoma transcript 1 (MALATI), ENE from multiple endocrine neoplasia p (MENP), etc.
Double Element for Nuclear Expression (dENE): containing two URILs separated by a predicated double helical region, examples include rice TWIFB1 dENE, and its 20 mutants (M1 to M20), which are known in the art and can be found described in FIG. 5 in Torabi et al., “RNA stabilization by a poly(A) tail 3′-end binding pocket and other modes of poly(A)-RNA interaction”, Science, 2021, 371(6529).
KSHV ENE sequence:

[SEQ ID NO: 4]
UGUUUUGGCUGGGUUUUUCCUUGUUCGCACCGGACACCUCCAGUGACCAG
ACGGCAAGGUUUUUAUCCCAGUGUAUAUU

Rrhesus rhadinovirus (PRV) ENE sequence:

[SEQ ID NO: 5]
CGUUUGUGUUGGUUUUUAUGACCAGCUUGGUACAAAACCUGCUGGUGAUU
UUUUACCCAACAAAUAAUAAAUAAAA

MALATI ENE sequence:

[SEQ ID NO: 6]
UAGGGUCAUGAAGGUUUUUCUUUUCCUGAGAAAACAACACGUAUUGUUUU
CUCAGGUUUUGCUUUUUGGCCUUUUUCUAGCUU

MALATI ENE+A-rich tract sequence:

[SEQ ID NO: 7]
UAGGGUCAUGAAGGUUUUUCUUUUCCUGAGAAAACAACACGUAUUGUUUU
CUCAGGUUUUGCUUUUUGGCCUUUUUCUAGCUUAAAAAAAAAAAAAGCAA
AA

MALATI ENE+A-rich tract+mascRNA sequence:

[SEQ ID NO: 8]
UAGGGUCAUGAAGGUUUUUCUUUUCCUGAGAAAACAACACGUAUUGUUUU
CUCAGGUUUUGCUUUUUGGCCUUUUUCUAGCUUAAAAAAAAAAAAAGCAA
AAGAUGCUGGUGGUUGGCACUCCUGGUUUCCAGGACGGGGUUCAAAUCCC
UGCGGCGUCUUUGCUUUGACU

MALATI ENE+A-rich tract variant sequence:

[SEQ ID NO: 9]
GAAGGUUUUUCUUUUCCUGAGAAAACAACACGUAUUGUUUUCUCAGGUUU
UGCUUUUUGGCCUUUUUCUAGCUUAAAAAAAAAAAAAGCAAAA

MENP ENE sequence:

[SEQ ID NO: 10]
GCCGCCGCAGGUGUUUCUUUUACUGAGUGCAGCCCAUGGCCGCACUCAGG
UUUUGCUUUUCACCUUCCCAUCUG

MENP ENE+A-rich tract sequence:

[SEQ ID NO: 11]
GCCGCCGCAGGUGUUUCUUUUACUGAGUGCAGCCCAUGGCCGCACUCAGG
UUUUGCUUUUCACCUUCCCAUCUGUGAAAGAGUGAGCAGGAAAAAGCAAA
A

MENP ENE+A-rich tract variant sequence:

[SEQ ID NO: 12]
AGGUGUUUCUUUUACUGAGUGCAGCCCAUGGCCGCACUCAGGUUUUGCUU
UUCACCUUCCCAUCUGUGAAAGAGUGAGCAGGAAAAAGCAAAA

Rice TWIFB1 dENE sequence:

[SEQ ID NO: 13]
UGUUGGCUGUACUCUUUUCUUUGUCAUGGUUUUCUCAAAUAUGAGUUUUU
ACAUGACAAAGUUUUUAACGAGGCAGCAUGUA.

MCDiV ENE sequence:

[SEQ ID NO: 14]
GAGUGUAACUCAACAGUUUUUCCUAACCACGCGUCGCGUGGCAGGUUUUU
UAAUCUGAGAGUUACAUUC

ATCOPIA27_ATh-I ENE sequence:

[SEQ ID NO: 15]
GUGCUGUACUCUUUUUCCUCACUAUGGUUUUGUCCCGAAAGGGUUUUCCU
AGUAAGGUUUUAAUGAGGCAGCAU

TUCP_ZMa ENE sequence:

[SEQ ID NO: 16]
GGCUGUACUCUUUUUUCCUGUCUAGGGUUUCUCACAAGGGUGAGUUUUAC
CUAGACAGGUUUUUAACGAGGCAACC

Other ENEs or dENEs and their variants or mutants are known in the art and can be found described in Tycowski et al., “Conservation of a Triple-Helix-Forming RNA Stability Element in Noncoding and Genomic RNAs of Diverse Viruses”, Cell Rep., 2012, 2: 26-32, and Tycowski et al., “Myriad Triple-Helix-Forming Structures in the Transposable Element RNAs of Plants and Fungi”, Cell Rep., 2016, 15: 1266-1276.
Some computational framework (e.g. TEISER, Tool for Eliciting Informative Structural Elements in RNA)) was used to identify structural RNA stability motif 1 (sRSM1), the most statistically significant 3′ UTR element that stabilizes RNA, which is known in the art and can be found described in Goodarzi et al., “Systematic discovery of structural elements governing stability of mammalian messenger RNAs”, Nature, 2012, 485(264).
Structural RNA stability motif 1 (sRSM1) sequence set 1:

[SEQ ID NO: 17]
AAAACUAUUUUGAAGAUGGUGGUGAGCUGCAAAAUAGCUGGAUGGAUUUG
AAUGAUUGGGAUGAUACAUCAUUGAACUGCACUUUAUAUAACCAAAGCUU
AGCAGUUUGUUAGAUAAGAGUCUAUGUAUGUCUCUGGUUAGGAUGAAGUU
AAUUUUAUGUUUUUAACAUGGUAUUUUUGAAGGAGCUAAUGAAACACUGG

Structural RNA stability motif 1 (sRSM1) sequence set 2:

[SEQ ID NO: 18]
AUUGUUUCUGGAAACUGCUUGCCAAGACAACAUUUAUUAACUGUUAGAAC
ACUUGCUUUAUGUUUGUGUGUACAUAUUUUCCACAAAUGUUAUAAUUUAU
AUAGUGUGGUUGAACAGGAUGCAAUCUUUUGUUGUCUAAAGGUGCUGCAG
UUAAAAAAAAAACAACCUUUUCUUUCAAUAUGGCAUGUAGUGGAGUUUUU

Other sRSM sequences are known to those of skill in the art, examples of which can be found in Goodarzi et al., “Systematic discovery of structural elements governing stability of mammalian messenger RNAs”, Nature, 2012, 485: 264.

Other suitable 3′UTR sequences are known to one of ordinary skill in the art and include, but are not limited to, c-fos gene and v-fos gene 3′UTR, CD47 3′UTR, BIRC3 3′UTR, beta-actin 3′UTR, beta-globin 3′UTR, Hmga2 3′ UTR, Camk2a 3′UTR, Cyclin B1 3′UTR and U-rich motifs that are associated with increased mRNA stability.

Other cis-acting regulatory elements are known to one of ordinary skill in the art and are incorporated herein. One of skill in the art, armed with the teachings of this specification, will be able identify and optimize suitable cis-acting regulatory elements without undue experimentation.

### CRISPR/Cas Proteins and Systems

Understanding the present invention will be aided by understanding CRISPER/Cas protein systems in general and in the context of the present invention.

### Prime-Editing Guide RNA

As discussed, above, several variations of prime editors (PEs) have been developed. The PE comprises a reverse transcriptase fused to an RNA-programmable nickase and a prime editing guide RNA to copy genetic information directly from an extension on the pegRNA into the target genomic locus. Thus, the pegRNA “steers” the PE editing apparatus to a specific site, the target DNA, where a single strand of the double-strand DNA is snipped by the Cas9 enzyme. The pegRNA also comprises the sequence the encodes the desired edit to the target DNA. Depending on the design of the pegRNA, PE can precisely and efficiently swap any single letter of DNA for any other, and can make both deletions and insertions. One of ordinary skill in the art understands how to construct a suitable pegRNA for a specific target site.

### RNA-Guided Endonucleases

RNA-guided endonucleases, such as Cas9, may comprise at least one nuclear localization signal, at least one nuclease domain, and at least one domain that interacts with a pegRNA to target the endonuclease to a specific nucleotide sequence for cleavage. Also known are nucleic acids encoding the RNA-guided endonucleases, as well as methods of using the RNA-guided endonucleases to modify chromosomal sequences of eukaryotic cells or embryos. The RNA-guided endonuclease interacts with specific pegRNAs, each of which directs the endonuclease to a specific targeted site, at which site the RNA-guided endonuclease introduces a stranded break that can be repaired by a DNA repair process such that the chromosomal sequence is modified. Since the specificity is provided by the pegRNA, the RNA-based endonuclease is universal and can be used with different pegRNAs to target different genomic sequences. The methods disclosed herein can be used to target and modify specific chromosomal sequences and/or introduce exogenous sequences (or delete endogenous sequences) at targeted locations in the genome of cells or embryos. Furthermore, the targeting is specific with limited off target effects.

The present disclosure provides fusion proteins, wherein a fusion protein comprises a CRISPR/Cas-like protein or fragment thereof and an effector domain. Suitable effector domains include, without limit, cleavage domains, epigenetic modification domains, transcriptional activation domains, and transcriptional repressor domains. Each fusion protein is guided to a specific chromosomal sequence by a specific pegRNA, wherein the effector domain mediates targeted genome modification or gene regulation. In one aspect, the fusion proteins can function as dimers thereby increasing the length of the target site and increasing the likelihood of its uniqueness in the genome (thus, reducing off target effects). For example, endogenous CRISPR systems modify genomic locations based on DNA binding word lengths of approximately 13-20 bp (Cong, et al., Science, 339:819-823). At this word size, only 5-7% of the target sites are unique within the genome (Iseli, et al., PLos One 2(6):e579). In contrast, DNA binding word sizes for zinc finger nucleases typically range from 30-36 bp, resulting in target sites that are approximately 85-87% unique within the human genome. The smaller sized DNA binding sites utilized by CRISPR-based systems limits and complicates design of targeted CRISP-based nucleases near desired locations, such as disease SNPs, small exons, start codons, and stop codons, as well as other locations within complex genomes. The present disclosure not only provides means for expanding the CRISPR DNA binding word length (i.e., so as to limit off-target activity), but further provides CRISPR fusion proteins having modified functionality. According, the disclosed CRISPR fusion proteins have increased target specificity and unique functionality(ies). Also provided herein are methods of using the fusion proteins to modify or regulate expression of targeted chromosomal sequences.

RNA-guided endonucleases may comprise at least one nuclear localization signal, which permits entry of the endonuclease into the nuclei of eukaryotic cells and embryos such as, for example, non-human one cell embryos. RNA-guided endonucleases also comprise at least one nuclease domain and at least one domain that interacts with a pegRNA. An RNA-guided endonuclease is directed to a specific nucleic acid sequence (or target site) by a pegRNA. The pegRNA interacts with the RNA-guided endonuclease as well as the target site such that, once directed to the target site, the RNA-guided endonuclease is able to introduce a stranded break into the target site nucleic acid sequence. Since the pegRNA provides the specificity for the targeted cleavage, the endonuclease of the RNA-guided endonuclease is universal and can be used with different pegRNAs to cleave different target nucleic acid sequences. RNA-guided endonucleases can be proteins, can be encoded by isolated nucleic acids (i.e., RNA or DNA), can be encoded by vectors comprising nucleic acids encoding the RNA-guided endonucleases, and can be protein-RNA complexes comprising the RNA-guided endonuclease plus a pegRNA.

The RNA-guided endonuclease can be derived from a clustered regularly interspersed short palindromic repeats (CRISPR)/CRISPR-associated (Cas) system. The CRISPR/Cas system can be a type I, a type II, or a type 11 system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the RNA-guided endonuclease is derived from a type II CRISPR/Cas system. In specific embodiments, the RNA-guided endonuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina.

In general, CRISPR/Cas proteins comprise at least one RNA recognition and/or RNA binding domain. RNA recognition and/or RNA binding domains interact with guide RNAs. CRISPR/Cas proteins can also comprise nuclease domains (i.e., DNase or RNase domains), DNA binding domains, helicase domains, RNAse domains, protein-protein interaction domains, dimerization domains, as well as other domains.

The CRISPR/Cas-like protein can be a wild type CRISPR/Cas protein, a modified CRISPR/Cas protein, or a fragment of a wild type or modified CRISPR/Cas protein. The CRISPR/Cas-like protein can be modified to increase nucleic acid binding affinity and/or specificity, alter an enzymatic activity, and/or change another property of the protein. For example, nuclease (i.e., DNase, RNase) domains of the CRISPR/Cas-like protein can be modified, deleted, or inactivated. Alternatively, the CRISPR/Cas-like protein can be truncated to remove domains that are not essential for the function of the fusion protein. The CRISPR/Cas-like protein can also be truncated or modified to optimize the activity of the effector domain of the fusion protein.

In some embodiments, the CRISPR/Cas-like protein can be derived from a wild type Cas9 protein or fragment thereof. In other embodiments, the CRISPR/Cas-like protein can be derived from modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek et al., Science, 337: 816-821). In some embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. For example, an aspartate to alanine (D10A) conversion in a RuvC-like domain converts the Cas9-derived protein into a nickase. Likewise, a histidine to alanine (H840A or H839A) conversion in a HNH domain converts the Cas9-derived protein into a nickase. Each nuclease domain can be modified using well-known methods, such as site-directed mutagenesis, PCR-mediated mutagenesis, and total gene synthesis, as well as other methods known in the art.

The RNA-guided endonuclease may comprise at least one nuclear localization signal. In general, an NLS comprises a stretch of basic amino acids. Nuclear localization signals are known in the art (see, e.g., Lange et al., J. Biol. Chem., 2007, 282:5101-5105). For example, in one embodiment, the NLS can be a monopartite sequence, such as PKKKRKV (SEQ ID NO:19) or PKKKRRV (SEQ ID NO:8). In another embodiment, the NLS can be a bipartite sequence. In still another embodiment, the NLS can be KRPAATKKAGQAKKKK (SEQ ID NO:20). The NLS can be located at the N-terminus, the C-terminal, or in an internal location of the RNA-guided endonuclease.

In some embodiments, the RNA-guided endonuclease can further comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO:21). In another embodiment, the cell-penetrating domain can be TLM (PLSSIFSRIGDPPKKKRKV; SEQ ID NO:22), a cell-penetrating peptide sequence derived from the human hepatitis B virus. In still another embodiment, the cell-penetrating domain can be MPG (GALFLGWLGAAGSTMGAPKKKRKV; SEQ ID NO:23 or GALFLGFLGAAGSTMGAWSQPKKKRKV; SEQ ID NO:24). In an additional embodiment, the cell-penetrating domain can be Pep-1 (KETWWETWWTEWSQPKKKRKV; SEQ ID NO:25), VP22, a cell penetrating peptide from Herpes simplex virus, or a polyarginine peptide sequence. The cell-penetrating domain can be located at the N-terminus, the C-terminus, or in an internal location of the protein.

In still other embodiments, the RNA-guided endonuclease can also comprise at least one marker domain. Non-limiting examples of marker domains include fluorescent proteins, purification tags, and epitope tags. In some embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire), cyan fluorescent proteins (e.g., ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6.times. His, biotin carboxyl carrier protein (BCCP), and calmodulin.

In certain embodiments, the RNA-guided endonuclease may be part of a protein-RNA complex comprising a pegRNA. The pegRNA interacts with the RNA-guided endonuclease to direct the endonuclease to a specific target site, wherein the 5′ end of the guide RNA base pairs with a specific protospacer sequence.

### (II) Fusion Proteins

Another aspect of the present disclosure provides a fusion protein comprising a CRISPR/Cas-like protein or fragment thereof and an effector domain in combination with a pegRNA and a cis-acting regulatory element. The CRISPR/Cas-like protein is directed to a target site by a pegRNA, at which site the effector domain can modify or effect the targeted nucleic acid sequence. The effector domain can be a cleavage domain, an epigenetic modification domain, a transcriptional activation domain, or a transcriptional repressor domain. The fusion protein can further comprise at least one additional domain chosen from a nuclear localization signal, a cell-penetrating domain, or a marker domain.

### (a) CRISPR/Cas-Like Protein

The fusion protein comprises a CRISPR/Cas-like protein or a fragment thereof. CRISPR/Cas-like proteins are detailed above in section (I). The CRISPR/Cas-like protein can be located at the N-terminus, the C-terminus, or in an internal location of the fusion protein

In some embodiments, the CRISPR/Cas-like protein of the fusion protein can be derived from a Cas9 protein. The Cas9-derived protein can be wild type, modified, or a fragment thereof. In some embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. For example, an aspartate to alanine (D10A) conversion in a RuvC-like domain converts the Cas9-derived protein into a nickase. Likewise, a histidine to alanine (H840A or H839A) conversion in a HNH domain converts the Cas9-derived protein into a nickase. In other embodiments, both of the RuvC-like nuclease domain and the HNH-like nuclease domain can be modified or eliminated such that the Cas9-derived protein is unable to nick or cleave double stranded nucleic acid. In still other embodiments, all nuclease domains of the Cas9-derived protein can be modified or eliminated such that the Cas9-derived protein lacks all nuclease activity.

In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art. In an exemplary embodiment, the CRISPR/Cas-like protein of the fusion protein is derived from a Cas9 protein in which all the nuclease domains have been inactivated or deleted.

### (b) Effector Domain

The fusion protein also comprises an effector domain. The effector domain can be a cleavage domain, an epigenetic modification domain, a transcriptional activation domain, or a transcriptional repressor domain. The effector domain can be located at the N-terminus, the C-terminus, or in an internal location of the fusion protein.

### (i) Cleavage Domain

In some embodiments, the effector domain is a cleavage domain. As used herein, a “cleavage domain” refers to a domain that cleaves DNA. The cleavage domain can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort, et al., (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). See also Linn, et al., (eds.) Nucleases, Cold Spring Harbor Laboratory Press, 1993. One or more of these enzymes (or functional fragments thereof) can be used as a source of cleavage domains.

In some embodiments, the cleavage domain can be derived from a type II-S endonuclease. Type II-S endonucleases cleave DNA at sites that are typically several base pairs away the recognition site and, as such, have separable recognition and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MbolI, and SapI. In exemplary embodiments, the cleavage domain of the fusion protein is a FokI cleavage domain or a derivative thereof.

In certain embodiments, the type II-S cleavage can be modified to facilitate dimerization of two different cleavage domains (each of which is attached to a CRISPR/Cas-like protein or fragment thereof). For example, the cleavage domain of FokI can be modified by mutating certain amino acid residues. By way of non-limiting example, amino acid residues at positions 446, 447, 479, 483, 484, 486, 487, 490, 491, 496, 498, 499, 500, 531, 534, 537, and 538 of FokI cleavage domains are targets for modification. For example, modified cleavage domains of FokI that form obligate heterodimers include a pair in which a first modified cleavage domain includes mutations at amino acid positions 490 and 538 and a second modified cleavage domain that includes mutations at amino acid positions 486 and 499 (Miller et al., 2007, Nat. Biotechnol, 25:778-785; Szczpek et al., 2007, Nat. Biotechnol, 25:786-793). For example, the Glu (E) at position 490 can be changed to Lys (K) and the IIe (I) at position 538 can be changed to K in one domain (E490K, 1538K), and the Gln (Q) at position 486 can be changed to E and the I at position 499 can be changed to Leu (L) in another cleavage domain (Q486E, 1499L). In other embodiments, modified FokI cleavage domains can include three amino acid changes (Doyon et al., 2011, Nat. Methods, 8:74-81). For example, one modified FokI domain (which is termed ELD) can comprise Q486E, 1499L, N496D mutations and the other modified FokI domain (which is termed KKR) can comprise E490K, 1538K, H537R mutations.

In exemplary embodiments, the effector domain of the fusion protein is a FokI cleavage domain or a modified FokI cleavage domain.

In embodiments wherein the effector domain is a cleavage domain and the CRISPR/Cas-like protein is derived from a Cas9 protein, the Cas9-derived can be modified as discussed herein such that its endonuclease activity is eliminated. For example, the Cas9-derived can be modified by mutating the RuvC and HNH domains such that they no longer possess nuclease activity.

### (ii) Epigenetic Modification Domain

In other embodiments, the effector domain of the fusion protein can be an epigenetic modification domain. In general, epigenetic modification domains alter histone structure and/or chromosomal structure without altering the DNA sequence. Changes histone and/or chromatin structure can lead to changes in gene expression. Examples of epigenetic modification include, without limit, acetylation or methylation of lysine residues in histone proteins, and methylation of cytosine residues in DNA. Non-limiting examples of suitable epigenetic modification domains include histone acetyltansferase domains, histone deacetylase domains, histone methyltransferase domains, histone demethylase domains, DNA methyltransferase domains, and DNA demethylase domains.

In embodiments in which the effector domain is a histone acetyltansferase (HAT) domain, the HAT domain can be derived from EP300 (i.e., E1A binding protein p300), CREBBP (i.e., CREB-binding protein), CDY1, CDY2, CDYL1, CLOCK, ELP3, ESA1, GCN5 (KAT2A), HAT1, KAT2B, KAT5, MYST1, MYST2, MYST3, MYST4, NCOA1, NCOA2, NCOA3, NCOAT, P/CAF, Tip60, TAFII250, or TF3C4. In one such embodiment, the HAT domain is p300

In embodiments wherein the effector domain is an epigenetic modification domain and the CRISPR/Cas-like protein is derived from a Cas9 protein, the Cas9-derived can be modified as discussed herein such that its endonuclease activity is eliminated. For example, the Cas9-derived can be modified by mutating the RuvC and HNH domains such that they no longer possess nuclease activity.

(iii) Transcriptional Activation Domain

In other embodiments, the effector domain of the fusion protein can be a transcriptional activation domain. In general, a transcriptional activation domain interacts with transcriptional control elements and/or transcriptional regulatory proteins (i.e., transcription factors, RNA polymerases, etc.) to increase and/or activate transcription of a gene. In some embodiments, the transcriptional activation domain can be, without limit, a herpes simplex virus VP16 activation domain, VP64 (which is a tetrameric derivative of VP16), a NF-κB p65 activation domain, p53 activation domains 1 and 2, a CREB (cAMP response element binding protein) activation domain, an E2A activation domain, and an NFAT (nuclear factor of activated T-cells) activation domain. In other embodiments, the transcriptional activation domain can be Gal4, Gcn4, MLL, Rtg3, Gln3, Oaf1, Pip2, Pdr1, Pdr3, Pho4, and Leu3. The transcriptional activation domain may be wild type, or it may be a modified version of the original transcriptional activation domain. In some embodiments, the effector domain of the fusion protein is a VP16 or VP64 transcriptional activation domain.

In embodiments wherein the effector domain is a transcriptional activation domain and the CRISPR/Cas-like protein is derived from a Cas9 protein, the Cas9-derived protein can be modified as discussed herein such that its endonuclease activity is eliminated. For example, the Cas9-derived can be modified by mutating the RuvC and HNH domains such that they no longer possess nuclease activity.

### (iv) Transcriptional Repressor Domain

In still other embodiments, the effector domain of the fusion protein can be a transcriptional repressor domain. In general, a transcriptional repressor domain interacts with transcriptional control elements and/or transcriptional regulatory proteins (i.e., transcription factors, RNA polymerases, etc.) to decrease and/or terminate transcription of a gene. Non-limiting examples of suitable transcriptional repressor domains include inducible cAMP early repressor (ICER) domains, Kruppel-associated box A (KRAB-A) repressor domains, YY1 glycine rich repressor domains, Sp1-like repressors, E(spl) repressors, IκB repressor, and MeCP2.

In embodiments wherein the effector domain is a transcriptional repressor domain and the CRISPR/Cas-like protein is derived from a Cas9 protein, the Cas9-derived protein can be modified as discussed herein such that its endonuclease activity is eliminated. For example, the cas9 can be modified by mutating the RuvC and HNH domains such that they no longer possess nuclease activity.

### (c) Additional Domains

In some embodiments, the fusion protein further comprises at least one additional domain. Non-limiting examples of suitable additional domains include nuclear localization signals, cell-penetrating or translocation domains, and marker domains. Non-limiting examples of suitable nuclear localization signals, cell-penetrating domains, and marker domains are presented above in section (1).

### (d) Fusion Protein Dimers

In embodiments in which the effector domain of the fusion protein is a cleavage domain, a dimer comprising at least one fusion protein can form. The dimer can be a homodimer or a heterodimer. In some embodiments, the heterodimer comprises two different fusion proteins. In other embodiments, the heterodimer comprises one fusion protein and an additional protein.

In some embodiments, the dimer is a homodimer in which the two fusion protein monomers are identical with respect to the primary amino acid sequence. In one embodiment where the dimer is a homodimer, the Cas9-derived proteins are modified such that their endonuclease activity is eliminated, i.e., such that they have no functional nuclease domains. In certain embodiments wherein the Cas9-derived proteins are modified such that their endonuclease activity is eliminated, each fusion protein monomer comprises an identical Cas9 like protein and an identical cleavage domain. The cleavage domain can be any cleavage domain, such as any of the exemplary cleavage domains provided herein. In one specific embodiment, the cleavage domain is a FokI cleavage domain or a modified FokI cleavage domain. In such embodiments, specific pegRNAs would direct the fusion protein monomers to different but closely adjacent sites such that, upon dimer formation, the nuclease domains of the two monomers would create a double stranded break in the target DNA.

In other embodiments, the dimer is a heterodimer of two different fusion proteins. For example, the CRISPR/Cas-like protein of each fusion protein can be derived from a different CRISPR/Cas protein or from an orthologous CRISPR/Cas protein from a different bacterial species. For example, each fusion protein can comprise a Cas9-like protein, which Cas9-like protein is derived from a different bacterial species. In these embodiments, each fusion protein would recognize a different target site (i.e., specified by the protospacer and/or PAM sequence). For example, the pegRNAs could position the heterodimer to different but closely adjacent sites such that their nuclease domains results in an effective double stranded break in the target DNA. The heterodimer can also have modified Cas9 proteins with nicking activity such that the nicking locations are different.

Alternatively, two fusion proteins of a heterodimer can have different effector domains. In embodiments in which the effector domain is a cleavage domain, each fusion protein can contain a different modified cleavage domain. For example, each fusion protein can contain a different modified FokI cleavage domain, as detailed above in section (II)(b)(i). In these embodiments, the Cas-9 proteins can be modified such that their endonuclease activities are eliminated.

As will be appreciated by those skilled in the art, the two fusion proteins forming a heterodimer can differ in both the CRISPR/Cas-like protein domain and the effector domain.

In any of the above-described embodiments, the homodimer or heterodimer can comprise at least one additional domain chosen from nuclear localization signals (NLSs), cell-penetrating, translocation domains and marker domains, as detailed above.

In any of the above-described embodiments, one or both of the Cas9-derived proteins can be modified such that its endonuclease activity is eliminated or modified.

In still alternate embodiments, the heterodimer comprises one fusion protein and an additional protein. For example, the additional protein can be a nuclease. In one embodiment, the nuclease is a zinc finger nuclease. A zinc finger nuclease comprises a zinc finger DNA binding domain and a cleavage domain. A zinc finger recognizes and binds three (3) nucleotides. A zinc finger DNA binding domain can comprise from about three zinc fingers to about seven zinc fingers. The zinc finger DNA binding domain can be derived from a naturally occurring protein or it can be engineered. See, for example, Beerli, et al., (2002) Nat. Biotechnol. 20:135-141; Pabo, et al., (2001) Ann. Rev. Biochem. 70:313-340; Isalan, et al., (2001) Nat. Biotechnol. 19:656-660; Segal et al. (2001) Curr. Opin. Biotechnol. 12:632-637; Choo, et al., (2000) Curr. Opin. Struct. Biol. 10:411-416; Zhang, et al., (2000) J. Biol. Chem. 275(43):33850-33860; Doyon, et al., (2008) Nat. Biotechnol. 26:702-708; and Santiago, et al., (2008) Proc. Natl. Acad. Sci. USA 105:5809-5814. The cleavage domain of the zinc finger nuclease can be any cleavage domain detailed above in section (II)(b)(i). In exemplary embodiments, the cleavage domain of the zinc finger nuclease is a FokI cleavage domain or a modified FokI cleavage domain. Such a zinc finger nuclease will dimerize with a fusion protein comprising a FokI cleavage domain or a modified FokI cleavage domain.

In some embodiments, the zinc finger nuclease can comprise at least one additional domain chosen from nuclear localization signals, cell-penetrating or translocation domains, which are detailed above.

In certain embodiments, any of the fusion protein detailed above or a dimer comprising at least one fusion protein may be part of a protein-RNA complex comprising at least one pegRNA. A pegRNA interacts with the CRISPR-Cas0 like protein of the fusion protein to direct the fusion protein to a specific target site, wherein the 5′ end of the pegRNA base pairs with a specific protospacer sequence.

### (III) Nucleic Acids Encoding RNA-Guided Endonucleases or Fusion Proteins

Another aspect of the present disclosure provides nucleic acids encoding any of the RNA-guided endonucleases or fusion proteins described above in sections (I) and (II), respectively. The nucleic acid can be RNA or DNA. In one embodiment, the nucleic acid encoding the RNA-guided endonuclease or fusion protein is mRNA. The mRNA can be 5′ capped and/or 3′ polyadenylated. In another embodiment, the nucleic acid encoding the RNA-guided endonuclease or fusion protein is DNA. The DNA can be present in a vector (see below).

The nucleic acid encoding the RNA-guided endonuclease or fusion protein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth. Programs for codon optimization are available as freeware. Commercial codon optimization programs are also available.

In some embodiments, DNA encoding the RNA-guided endonuclease or fusion protein can be operably linked to at least one promoter control sequence. In some iterations, the DNA coding sequence can be operably linked to a promoter control sequence for expression in the eukaryotic cell or animal of interest. The promoter control sequence can be constitutive, regulated, or tissue-specific. Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, Nphsl promoter, OG-2 promoter, SP-B promoter, SYN1 promoter, and WASP promoter. The promoter sequence can be wild type or it can be modified for more efficient or efficacious expression. In one exemplary embodiment, the encoding DNA can be operably linked to a CMV promoter for constitutive expression in mammalian cells.

In certain embodiments, the sequence encoding the RNA-guided endonuclease or fusion protein can be operably linked to a promoter sequence that is recognized by a phage RNA polymerase for in vitro mRNA synthesis. In such embodiments, the in vitro-transcribed RNA can be purified for use in the methods detailed below in sections (IV) and (V). For example, the promoter sequence can be a T7, T3, or SP6 promoter sequence or a variation of a T7, T3, or SP6 promoter sequence. In an exemplary embodiment, the DNA encoding the fusion protein is operably linked to a T7 promoter for in vitro mRNA synthesis using T7 RNA polymerase.

In alternate embodiments, the sequence encoding the RNA-guided endonuclease or fusion protein can be operably linked to a promoter sequence for in vitro expression of the RNA-guided endonuclease or fusion protein in bacterial or eukaryotic cells. In such embodiments, the expressed protein can be purified for use in the methods detailed below in sections (IV) and (V). Suitable bacterial promoters include, without limit, T7 promoters, lac operon promoters, trp promoters, variations thereof, and combinations thereof. An exemplary bacterial promoter is tac which is a hybrid of trp and lac promoters. Non-limiting examples of suitable eukaryotic promoters are listed above.

In additional aspects, the DNA encoding the RNA-guided endonuclease or fusion protein also can be linked to a polyadenylation signal (e.g., SV40 polyA signal, bovine growth hormone (BGH) polyA signal, etc.) and/or at least one transcriptional termination sequence. Additionally, the sequence encoding the RNA-guided endonuclease or fusion protein also can be linked to sequence encoding at least one nuclear localization signal, at least one cell-penetrating domain, and/or at least one marker domain, which are detailed above in section (I).

In various embodiments, the DNA encoding the RNA-guided endonuclease or fusion protein can be present in a vector. Suitable vectors include plasmid vectors, phagemids, cosmids, artificial/mini-chromosomes, transposons, and viral vectors (e.g., lentiviral vectors, adeno-associated viral vectors, etc.). In one embodiment, the DNA encoding the RNA-guided endonuclease or fusion protein is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel, et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In some embodiments, the expression vector comprising the sequence encoding the RNA-guided endonuclease or fusion protein can further comprise sequence encoding a pegRNA. The sequence encoding the pegRNA generally is operably linked to at least one transcriptional control sequence for expression of the pegRNA in the cell or embryo of interest. For example, DNA encoding the pegRNA can be operably linked to a promoter sequence that is recognized by RNA polymerase 11 (Pol Ill). Examples of suitable Pol Ill promoters include, but are not limited to, mammalian U6, U3, H1, and 7SL RNA promoters.

### (IV) Method for Modifying a Chromosomal Sequence Using an RNA-Guided Endonuclease

Another aspect of the present disclosure encompasses a method for modifying a chromosomal sequence in a eukaryotic cell or embryo. The method comprises introducing into a eukaryotic cell or embryo (i) at least one RNA-guided endonuclease comprising at least one nuclear localization signal or nucleic acid encoding at least one RNA-guided endonuclease comprising at least one nuclear localization signal, (ii) at least one pegRNA or DNA encoding at least one pegRNA, and, optionally, (iii) at least one donor polynucleotide comprising a donor sequence. The method further comprises culturing the cell or embryo such that each pegRNA directs an RNA-guided endonuclease to a targeted site in the chromosomal sequence where the RNA-guided endonuclease introduces a double-stranded break in the targeted site, and the double-stranded break is repaired by a DNA repair process such that the chromosomal sequence is modified.

In some embodiments, the method can comprise introducing one RNA-guided endonuclease (or encoding nucleic acid) and one pegRNA (or encoding DNA) into a cell or embryo, wherein the RNA-guided endonuclease introduces one double-stranded break in the targeted chromosomal sequence. In embodiments in which the optional donor polynucleotide is not present, the double-stranded break in the chromosomal sequence can be repaired by a non-homologous end-joining (NHEJ) repair process. Because NHEJ is error-prone, deletions of at least one nucleotide, insertions of at least one nucleotide, substitutions of at least one nucleotide, or combinations thereof can occur during the repair of the break. Accordingly, the targeted chromosomal sequence can be modified or inactivated. For example, a single nucleotide change (SNP) can give rise to an altered protein product, or a shift in the reading frame of a coding sequence can inactivate or “knock out” the sequence such that no protein product is made. In embodiments in which the optional donor polynucleotide is present, the donor sequence in the donor polynucleotide can be exchanged with or integrated into the chromosomal sequence at the targeted site during repair of the double-stranded break. For example, in embodiments in which the donor sequence is flanked by upstream and downstream sequences having substantial sequence identity with upstream and downstream sequences, respectively, of the targeted site in the chromosomal sequence, the donor sequence can be exchanged with or integrated into the chromosomal sequence at the targeted site during repair mediated by homology-directed repair process. Alternatively, in embodiments in which the donor sequence is flanked by compatible overhangs (or the compatible overhangs are generated in situ by the RNA-guided endonuclease) the donor sequence can be ligated directly with the cleaved chromosomal sequence by a non-homologous repair process during repair of the double-stranded break. Exchange or integration of the donor sequence into the chromosomal sequence modifies the targeted chromosomal sequence or introduces an exogenous sequence into the chromosomal sequence of the cell or embryo.

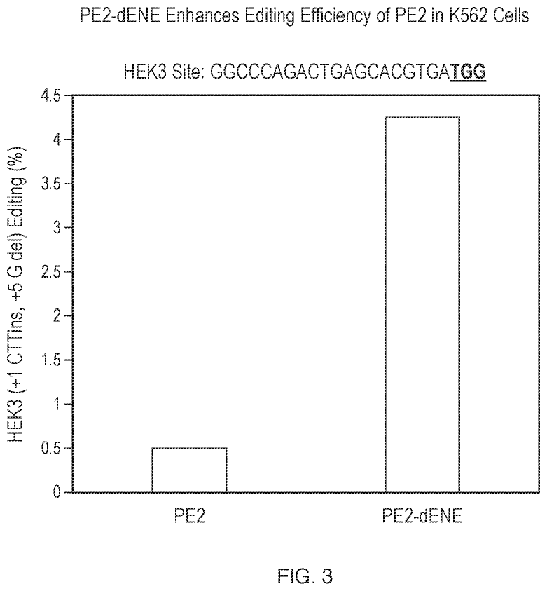

In other embodiments, the method can comprise introducing two RNA-guided endonucleases (or encoding nucleic acid) and two pegRNAs (or encoding DNA) into a cell or embryo, wherein the RNA-guided endonucleases introduce two double-stranded breaks in the chromosomal sequence. See FIG. 3B. The two breaks can be within several base pairs, within tens of base pairs, or can be separated by many thousands of base pairs. In embodiments in which the optional donor polynucleotide is not present, the resultant double-stranded breaks can be repaired by a non-homologous repair process such that the sequence between the two cleavage sites is lost and/or deletions of at least one nucleotide, insertions of at least one nucleotide, substitutions of at least one nucleotide, or combinations thereof can occur during the repair of the break(s). In embodiments in which the optional donor polynucleotide is present, the donor sequence in the donor polynucleotide can be exchanged with or integrated into the chromosomal sequence during repair of the double-stranded breaks by either a homology-based repair process (e.g., in embodiments in which the donor sequence is flanked by upstream and downstream sequences having substantial sequence identity with upstream and downstream sequences, respectively, of the targeted sites in the chromosomal sequence) or a non-homologous repair process (e.g., in embodiments in which the donor sequence is flanked by compatible overhangs).

In still other embodiments, the method can comprise introducing one RNA-guided endonuclease modified to cleave one strand of a double-stranded sequence (or encoding nucleic acid) and two pegRNAs (or encoding DNA) into a cell or embryo, wherein each pegRNA directs the RNA-guided endonuclease to a specific target site, at which site the modified endonuclease cleaves one strand (i.e., nicks) of the double-stranded chromosomal sequence, and wherein the two nicks are in opposite stands and in close enough proximity to constitute a double-stranded break. See FIG. 3A. In embodiments in which the optional donor polynucleotide is not present, the resultant double-stranded break can be repaired by a non-homologous repair process such that deletions of at least one nucleotide, insertions of at least one nucleotide, substitutions of at least one nucleotide, or combinations thereof can occur during the repair of the break. In embodiments in which the optional donor polynucleotide is present, the donor sequence in the donor polynucleotide can be exchanged with or integrated into the chromosomal sequence during repair of the double-stranded break by either a homology-based repair process (e.g., in embodiments in which the donor sequence is flanked by upstream and downstream sequences having substantial sequence identity with upstream and downstream sequences, respectively, of the targeted sites in the chromosomal sequence) or a non-homologous repair process (e.g., in embodiments in which the donor sequence is flanked by compatible overhangs).

### (a) RNA-Guided Endonuclease

The method comprises introducing into a cell or embryo at least one RNA-guided endonuclease comprising at least one nuclear localization signal or nucleic acid encoding at least one RNA-guided endonuclease comprising at least one nuclear localization signal. Such RNA-guided endonucleases and nucleic acids encoding RNA-guided endonucleases are described above in sections (1) and (111), respectively. Such RNA-guided may be pegRNA.

In some embodiments, the RNA-guided endonuclease can be introduced into the cell or embryo as an isolated protein. In such embodiments, the RNA-guided endonuclease can further comprise at least one cell-penetrating domain, which facilitates cellular uptake of the protein. In other embodiments, the RNA-guided endonuclease can be introduced into the cell or embryo as an mRNA molecule. In still other embodiments, the RNA-guided endonuclease can be introduced into the cell or embryo as a DNA molecule. In general, DNA sequence encoding the fusion protein is operably linked to a promoter sequence that will function in the cell or embryo of interest. The DNA sequence can be linear, or the DNA sequence can be part of a vector. In still other embodiments, the fusion protein can be introduced into the cell or embryo as an RNA-protein complex comprising the fusion protein and the pegRNA.

In alternate embodiments, DNA encoding the RNA-guided endonuclease can further comprise sequence encoding a pegRNA. In general, each of the sequences encoding the RNA-guided endonuclease and the pegRNA is operably linked to appropriate promoter control sequence that allows expression of the RNA-guided endonuclease and the pegRNA, respectively, in the cell or embryo. The DNA sequence encoding the RNA-guided endonuclease and the pegRNA can further comprise additional expression control, regulatory, and/or processing sequence(s). The DNA sequence encoding the RNA-guided endonuclease and the pegRNA can be linear or can be part of a vector

### (b) Prime Editing Guide RNA (PegRNA)

The method also comprises introducing into a cell or embryo at least one pegRNA or DNA encoding at least one pegRNA. A pegRNA interacts with the RNA-guided endonuclease to direct the endonuclease to a specific target site, at which site the 5′ end of the pegRNA base pairs with a specific protospacer sequence in the chromosomal sequence.

Each pegRNA comprises three regions: a first region at the 5′ end that is complementary to the target site in the chromosomal sequence, a second internal region that forms a stem loop structure, and a third 3′ region that remains essentially single-stranded. The first region of each pegRNA is different such that each pegRNA guides a fusion protein to a specific target site. The second and third regions of each pegRNA can be the same in all pegRNAs.

The first region of the pegRNA is complementary to sequence (i.e., protospacer sequence) at the target site in the chromosomal sequence such that the first region of the pegRNA can base pair with the target site. In various embodiments, the first region of the pegRNA can comprise from about 10 nucleotides to more than about 25 nucleotides. For example, the region of base pairing between the first region of the pegRNA and the target site in the chromosomal sequence can be about 10, 11, 12, 13, 14, 15, 16, 17, 18, 19, 20, 22, 23, 24, 25, or more than 25 nucleotides in length. In an exemplary embodiment, the first region of the pegRNA is about 19, 20, or 21 nucleotides in length.

The pegRNA also comprises a second region that forms a secondary structure. In some embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The pegRNA also comprises a third region at the 3′ end that remains essentially single-stranded. Thus, the third region has no complementarity to any chromosomal sequence in the cell of interest and has no complementarity to the rest of the pegRNA. The length of the third region can vary. In general, the third region is more than about 4 nucleotides in length. For example, the length of the third region can range from about 5 to about 60 nucleotides in length.

The combined length of the second and third regions (also called the universal or scaffold region) of the pegRNA can range from about 30 to about 120 nucleotides in length. In one aspect, the combined length of the second and third regions of the pegRNA range from about 70 to about 100 nucleotides in length.

In some embodiments, the pegRNA comprises a single molecule comprising all three regions. In other embodiments, the pegRNA can comprise two separate molecules. The first RNA molecule can comprise the first region of the pegRNA and one half of the “stem” of the second region of the pegRNA. The second RNA molecule can comprise the other half of the “stem” of the second region of the pegRNA and the third region of the pegRNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence to form a functional pegRNA.

In some embodiments, the pegRNA can be introduced into the cell or embryo as a RNA molecule. The RNA molecule can be transcribed in vitro. Alternatively, the RNA molecule can be chemically synthesized.

In other embodiments, the pegRNA can be introduced into the cell or embryo as a DNA molecule. In such cases, the DNA encoding the pegRNA can be operably linked to promoter control sequence for expression of the pegRNA in the cell or embryo of interest. For example, the RNA coding sequence can be operably linked to a promoter sequence that is recognized by RNA polymerase 11 (Pol Ill). Examples of suitable Pol Ill promoters include, but are not limited to, mammalian U6 or H1 promoters. In exemplary embodiments, the RNA coding sequence is linked to a mouse or human U6 promoter. In other exemplary embodiments, the RNA coding sequence is linked to a mouse or human H1 promoter.

The DNA molecule encoding the pegRNA can be linear or circular. In some embodiments, the DNA sequence encoding the pegRNA can be part of a vector. Suitable vectors include plasmid vectors, phagemids, cosmids, artificial/mini-chromosomes, transposons, and viral vectors. In an exemplary embodiment, the DNA encoding the RNA-guided endonuclease is present in a plasmid vector. Non-limiting examples of suitable plasmid vectors include pUC, pBR322, pET, pBluescript, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like.

In embodiments in which both the RNA-guided endonuclease and the pegRNA are introduced into the cell as DNA molecules, each can be part of a separate molecule (e.g., one vector containing fusion protein coding sequence and a second vector containing pegRNA coding sequence) or both can be part of the same molecule (e.g., one vector containing coding (and regulatory) sequence for both the fusion protein and the pegRNA).]

### (c) Target Site

An RNA-guided endonuclease in conjunction with a pegRNA is directed to a target site in the chromosomal sequence, wherein the RNA-guided endonuclease introduces a break in the chromosomal sequence. The target site has no sequence limitation except that the sequence is immediately followed (downstream) by a consensus sequence. This consensus sequence is also known as a protospacer adjacent motif (PAM). Examples of PAM include, but are not limited to, NGG, NGGNG, and NNAGAAW (wherein N is defined as any nucleotide and W is defined as either A or T). As detailed above in section (IV)(b), the first region (at the 5′ end) of the pegRNA is complementary to the protospacer of the target sequence. Typically, the first region of the pegRNA is about 19 to 21 nucleotides in length. Thus, in certain aspects, the sequence of the target site in the chromosomal sequence is 5′-N₁₉₋₂₁-NGG-3′. The PAM is in italics.

The target site can be in the coding region of a gene, in an intron of a gene, in a control region of a gene, in a non-coding region between genes, etc. The gene can be a protein coding gene or an RNA coding gene. The gene can be any gene of interest.

### (d) Optional Donor Polynucleotide

In some embodiments, the method further comprises introducing at least one donor polynucleotide into the target site. A donor polynucleotide comprises at least one donor sequence. In some aspects, a donor sequence of the donor polynucleotide corresponds to an endogenous or native chromosomal sequence. For example, the donor sequence can be essentially identical to a portion of the chromosomal sequence at or near the targeted site, but which comprises at least one nucleotide change. Thus, the donor sequence can comprise a modified version of the wild type sequence at the targeted site such that, upon integration or exchange with the native sequence, the sequence at the targeted chromosomal location comprises at least one nucleotide change. For example, the change can be an insertion of one or more nucleotides, a deletion of one or more nucleotides, a substitution of one or more nucleotides, or combinations thereof. As a consequence of the integration of the modified sequence, the cell or embryo/animal can produce a modified gene product from the targeted chromosomal sequence.

In other aspects, the donor sequence of the donor polynucleotide corresponds to an exogenous sequence. As used herein, an “exogenous” sequence refers to a sequence that is not native to the cell or embryo, or a sequence whose native location in the genome of the cell or embryo is in a different location. For example, the exogenous sequence can comprise protein coding sequence, which can be operably linked to an exogenous promoter control sequence such that, upon integration into the genome, the cell or embryo/animal is able to express the protein coded by the integrated sequence. Alternatively, the exogenous sequence can be integrated into the chromosomal sequence such that its expression is regulated by an endogenous promoter control sequence. In other iterations, the exogenous sequence can be a transcriptional control sequence, another expression control sequence, an RNA coding sequence, and so forth. Integration of an exogenous sequence into a chromosomal sequence is termed a “knock in.”

As can be appreciated by those skilled in the art, the length of the donor sequence can and will vary. For example, the donor sequence can vary in length from several nucleotides to hundreds of nucleotides to hundreds of thousands of nucleotides.

Donor polynucleotide comprising upstream and downstream sequences. In some embodiments, the donor sequence in the donor polynucleotide is flanked by an upstream sequence and a downstream sequence, which have substantial sequence identity to sequences located upstream and downstream, respectively, of the targeted site in the chromosomal sequence. Because of these sequence similarities, the upstream and downstream sequences of the donor polynucleotide permit homologous recombination between the donor polynucleotide and the targeted chromosomal sequence such that the donor sequence can be integrated into (or exchanged with) the chromosomal sequence.

The upstream sequence, as used herein, refers to a nucleic acid sequence that shares substantial sequence identity with a chromosomal sequence upstream of the targeted site. Similarly, the downstream sequence refers to a nucleic acid sequence that shares substantial sequence identity with a chromosomal sequence downstream of the targeted site. As used herein, the phrase “substantial sequence identity” refers to sequences having at least about 75% sequence identity. Thus, the upstream and downstream sequences in the donor polynucleotide can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with a sequence upstream or downstream to the targeted site. In an exemplary embodiment, the upstream and downstream sequences in the donor polynucleotide can have about 95% to 100% sequence identity with chromosomal sequences upstream or downstream to the targeted site. In one embodiment, the upstream sequence shares substantial sequence identity with a chromosomal sequence located immediately upstream of the targeted site (i.e., adjacent to the targeted site). In other embodiments, the upstream sequence shares substantial sequence identity with a chromosomal sequence that is located within about one hundred (100) nucleotides upstream from the targeted site. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the targeted site. In one embodiment, the downstream sequence shares substantial sequence identity with a chromosomal sequence located immediately downstream of the targeted site (i.e., adjacent to the targeted site). In other embodiments, the downstream sequence shares substantial sequence identity with a chromosomal sequence that is located within about one hundred (100) nucleotides downstream from the targeted site. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the targeted site.

Each upstream or downstream sequence can range in length from about 20 nucleotides to about 5000 nucleotides. In some embodiments, upstream and downstream sequences can comprise about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2800, 3000, 3200, 3400, 3600, 3800, 4000, 4200, 4400, 4600, 4800, or 5000 nucleotides. In exemplary embodiments, upstream and downstream sequences can range in length from about 50 to about 1500 nucleotides.

Donor polynucleotides comprising the upstream and downstream sequences with sequence similarity to the targeted chromosomal sequence can be linear or circular. In embodiments in which the donor polynucleotide is circular, it can be part of a vector. For example, the vector can be a plasmid vector.

Donor polynucleotide comprising targeted cleavage site(s). In other embodiments, the donor polynucleotide can additionally comprise at least one targeted cleavage site that is recognized by the RNA-guided endonuclease. The targeted cleavage site added to the donor polynucleotide can be placed upstream or downstream or both upstream and downstream of the donor sequence. For example, the donor sequence can be flanked by targeted cleavage sites such that, upon cleavage by the RNA-guided endonuclease, the donor sequence is flanked by overhangs that are compatible with those in the chromosomal sequence generated upon cleavage by the RNA-guided endonuclease. Accordingly, the donor sequence can be ligated with the cleaved chromosomal sequence during repair of the double stranded break by a non-homologous repair process. Generally, donor polynucleotides comprising the targeted cleavage site(s) will be circular (e.g., can be part of a plasmid vector).

Donor polynucleotide comprising a short donor sequence with optional overhangs. In still alternate embodiments, the donor polynucleotide can be a linear molecule comprising a short donor sequence with optional short overhangs that are compatible with the overhangs generated by the RNA-guided endonuclease. In such embodiments, the donor sequence can be ligated directly with the cleaved chromosomal sequence during repair of the double-stranded break. In some instances, the donor sequence can be less than about 1,000, less than about 500, less than about 250, or less than about 100 nucleotides. In certain cases, the donor polynucleotide can be a linear molecule comprising a short donor sequence with blunt ends. In other iterations, the donor polynucleotide can be a linear molecule comprising a short donor sequence with 5′ and/or 3′ overhangs. The overhangs can comprise 1, 2, 3, 4, or 5 nucleotides.

Typically, the donor polynucleotide will be DNA. The DNA may be single-stranded or double-stranded and/or linear or circular. The donor polynucleotide may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. In certain embodiments, the donor polynucleotide comprising the donor sequence can be part of a plasmid vector. In any of these situations, the donor polynucleotide comprising the donor sequence can further comprise at least one additional sequence.

### (e) Introducing Into the Cell or Embryo

The RNA-targeted endonuclease(s) (or encoding nucleic acid), the pegRNA(s) (or encoding DNA), and the optional donor polynucleotide(s) can be introduced into a cell or embryo by a variety of means. In some embodiments, the cell or embryo is transfected. Suitable transfection methods include calcium phosphate-mediated transfection, nucleofection (or electroporation), cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel, et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001). In other embodiments, the molecules are introduced into the cell or embryo by microinjection. Typically, the embryo is a fertilized one-cell stage embryo of the species of interest. For example, the molecules can be injected into the pronuclei of one cell embryos.

The RNA-targeted endonuclease(s) (or encoding nucleic acid), the pegRNA(s) (or DNAs encoding the pegRNA), and the optional donor polynucleotide(s) can be introduced into the cell or embryo simultaneously or sequentially. The ratio of the RNA-targeted endonuclease(s) (or encoding nucleic acid) to the pegRNA(s) (or encoding DNA) generally will be about stoichiometric such that they can form an RNA-protein complex. In one embodiment, DNA encoding an RNA-targeted endonuclease and DNA encoding a pegRNA are delivered together within the plasmid vector.

### (f) Culturing the Cell or Embryo

The method further comprises maintaining the cell or embryo under appropriate conditions such that the pegRNA(s) directs the RNA-guided endonuclease(s) to the targeted site(s) in the chromosomal sequence, and the RNA-guided endonuclease(s) introduce at least one double-stranded break in the chromosomal sequence. A double-stranded break can be repaired by a DNA repair process such that the chromosomal sequence is modified by a deletion of at least one nucleotide, an insertion of at least one nucleotide, a substitution of at least one nucleotide, or a combination thereof.

In embodiments in which no donor polynucleotide is introduced into the cell or embryo, the double-stranded break can be repaired via a non-homologous end-joining (NHEJ) repair process. Because NHEJ is error-prone, deletions of at least one nucleotide, insertions of at least one nucleotide, substitutions of at least one nucleotide, or combinations thereof can occur during the repair of the break. Accordingly, the sequence at the chromosomal sequence can be modified such that the reading frame of a coding region can be shifted and that the chromosomal sequence is inactivated or “knocked out.” An inactivated protein-coding chromosomal sequence does not give rise to the protein coded by the wild type chromosomal sequence.

In embodiments in which a donor polynucleotide comprising upstream and downstream sequences is introduced into the cell or embryo, the double-stranded break can be repaired by a homology-directed repair (HDR) process such that the donor sequence is integrated into the chromosomal sequence. Accordingly, an exogenous sequence can be integrated into the genome of the cell or embryo, or the targeted chromosomal sequence can be modified by exchange of a modified sequence for the wild type chromosomal sequence.

In embodiments in which a donor polynucleotide comprising the targeted cleave site is introduced into the cell or embryo, the RNA-guided endonuclease can cleave both the targeted chromosomal sequence and the donor polynucleotide. The linearized donor polynucleotide can be integrated into the chromosomal sequence at the site of the double-stranded break by ligation between the donor polynucleotide and the cleaved chromosomal sequence via a NHEJ process.

In embodiments in which a linear donor polynucleotide comprising a short donor sequence is introduced into the cell or embryo, the short donor sequence can be integrated into the chromosomal sequence at the site of the double-stranded break via a NHEJ process. The integration can proceed via the ligation of blunt ends between the short donor sequence and the chromosomal sequence at the site of the double stranded break. Alternatively, the integration can proceed via the ligation of sticky ends (i.e., having 5′ or 3′ overhangs) between a short donor sequence that is flanked by overhangs that are compatible with those generated by the RNA-targeting endonuclease in the cleaved chromosomal sequence.

In general, the cell is maintained under conditions appropriate for cell growth and/or maintenance. Suitable cell culture conditions are well known in the art and are described, for example, in Santiago, et al., (2008) PNAS 105:5809-5814; Moehle, et al., (2007) PNAS 104:3055-3060; Urnov, et al., (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

An embryo can be cultured in vitro (e.g., in cell culture). Typically, the embryo is cultured at an appropriate temperature and in appropriate media with the necessary O₂/CO₂ ratio to allow the expression of the RNA endonuclease and pegRNA, if necessary. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking, the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and can result in a live birth of an animal derived from the embryo. Such an animal would comprise the modified chromosomal sequence in every cell of the body.

### (g) Cell and Embryo Types

A variety of eukaryotic cells and embryos are suitable for use in the method. For example, the cell can be a human cell, a non-human mammalian cell, a non-mammalian vertebrate cell, an invertebrate cell, an insect cell, a plant cell, a yeast cell, or a single cell eukaryotic organism. In general, the embryo is non-human mammalian embryo. In specific embodiments, the embryos can be a one cell non-human mammalian embryo. Exemplary mammalian embryos, including one cell embryos, include without limit mouse, rat, hamster, rodent, rabbit, feline, canine, ovine, porcine, bovine, equine, and primate embryos. In still other embodiments, the cell can be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary embodiments, the cell is a mammalian cell.

Non-limiting examples of suitable mammalian cells include Chinese hamster ovary (CHO) cells, baby hamster kidney (BHK) cells; mouse myeloma NSO cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, and human K562 cells. An extensive list of mammalian cell lines may be found in the American Type Culture Collection catalog (ATCC, Manassas, Va.).

### (V) Method for Using a Fusion Protein to Modify a Chromosomal Sequence or Regulate Expression of a Chromosomal Sequence

Another aspect of the present disclosure encompasses a method for modifying a chromosomal sequence or regulating expression of a chromosomal sequence in a cell or embryo. The method comprises introducing into the cell or embryo (a) at least one fusion protein or nucleic acid encoding at least one fusion protein, wherein the fusion protein comprises a CRISPR/Cas-like protein or a fragment thereof and an effector domain, and (b) at least one pegRNA or DNA encoding the pegRNA, wherein the pegRNA guides the CRISPR/Cas-like protein of the fusion protein to a targeted site in the chromosomal sequence and the effector domain of the fusion protein modifies the chromosomal sequence or regulates expression of the chromosomal sequence.

Fusion proteins comprising a CRISPR/Cas-like protein or a fragment thereof and an effector domain are detailed above in section (II). In general, the fusion proteins disclosed herein further comprise at least one nuclear localization signal. Nucleic acids encoding fusion proteins are described above in section (III). In some embodiments, the fusion protein can be introduced into the cell or embryo as an isolated protein (which can further comprise a cell-penetrating domain). Furthermore, the isolated fusion protein can be part of a protein-RNA complex comprising the pegRNA. In other embodiments, the fusion protein can be introduced into the cell or embryo as a RNA molecule (which can be capped and/or polyadenylated). In still other embodiments, the fusion protein can be introduced into the cell or embryo as a DNA molecule. For example, the fusion protein and the pegRNA can be introduced into the cell or embryo as discrete DNA molecules or as part of the same DNA molecule. Such DNA molecules can be plasmid vectors.

In some embodiments, the method further comprises introducing into the cell or embryo at least one zinc finger nuclease. Zinc finger nucleases are described above in section (II)(d). In still other embodiments, the method further comprises introducing into the cell or embryo at least one donor polynucleotide. Donor polynucleotides are detailed above in section (IV)(d). Means for introducing molecules into cells or embryos, as well as means for culturing cell or embryos are described above in sections (IV)(e) and (IV)(f), respectively. Suitable cells and embryos are described above in section (IV)(g).

In certain embodiments in which the effector domain of the fusion protein is a cleavage domain (e.g., a FokI cleavage domain or a modified FokI cleavage domain), the method can comprise introducing into the cell or embryo one fusion protein (or nucleic acid encoding one fusion protein) and two pegRNAs (or DNA encoding two pegRNAs). The two pegRNAs direct the fusion protein to two different target sites in the chromosomal sequence, wherein the fusion protein dimerizes (e.g., form a homodimer) such that the two cleavage domains can introduce a double stranded break into the chromosomal sequence. In embodiments in which the optional donor polynucleotide is not present, the double-stranded break in the chromosomal sequence can be repaired by a non-homologous end-joining (NHEJ) repair process. Because NHEJ is error-prone, deletions of at least one nucleotide, insertions of at least one nucleotide, substitutions of at least one nucleotide, or combinations thereof can occur during the repair of the break. Accordingly, the targeted chromosomal sequence can be modified or inactivated. For example, a single nucleotide change (SNP) can give rise to an altered protein product, or a shift in the reading frame of a coding sequence can inactivate or “knock out” the sequence such that no protein product is made. In embodiments in which the optional donor polynucleotide is present, the donor sequence in the donor polynucleotide can be exchanged with or integrated into the chromosomal sequence at the targeted site during repair of the double-stranded break. For example, in embodiments in which the donor sequence is flanked by upstream and downstream sequences having substantial sequence identity with upstream and downstream sequences, respectively, of the targeted site in the chromosomal sequence, the donor sequence can be exchanged with or integrated into the chromosomal sequence at the targeted site during repair mediated by homology-directed repair process. Alternatively, in embodiments in which the donor sequence is flanked by compatible overhangs (or the compatible overhangs are generated in situ by the RNA-guided endonuclease) the donor sequence can be ligated directly with the cleaved chromosomal sequence by a non-homologous repair process during repair of the double-stranded break. Exchange or integration of the donor sequence into the chromosomal sequence modifies the targeted chromosomal sequence or introduces an exogenous sequence into the chromosomal sequence of the cell or embryo.

In other embodiments in which the effector domain of the fusion protein is a cleavage domain (e.g., a FokI cleavage domain or a modified FokI cleavage domain), the method can comprise introducing into the cell or embryo two different fusion proteins (or nucleic acid encoding two different fusion proteins) and two pegRNAs (or DNA encoding two pegRNAs). The fusion proteins can differ as detailed above in section (II). Each pegRNA directs a fusion protein to a specific target site in the chromosomal sequence, wherein the fusion proteins dimerize (e.g., form a heterodimer) such that the two cleavage domains can introduce a double stranded break into the chromosomal sequence. In embodiments in which the optional donor polynucleotide is not present, the resultant double-stranded breaks can be repaired by a non-homologous repair process such that deletions of at least one nucleotide, insertions of at least one nucleotide, substitutions of at least one nucleotide, or combinations thereof can occur during the repair of the break. In embodiments in which the optional donor polynucleotide is present, the donor sequence in the donor polynucleotide can be exchanged with or integrated into the chromosomal sequence during repair of the double-stranded break by either a homology-based repair process (e.g., in embodiments in which the donor sequence is flanked by upstream and downstream sequences having substantial sequence identity with upstream and downstream sequences, respectively, of the targeted sites in the chromosomal sequence) or a non-homologous repair process (e.g., in embodiments in which the donor sequence is flanked by compatible overhangs).

In still other embodiments in which the effector domain of the fusion protein is a cleavage domain (e.g., a FokI cleavage domain or a modified FokI cleavage domain), the method can comprise introducing into the cell or embryo one fusion protein (or nucleic acid encoding one fusion protein), one pegRNA (or DNA encoding one pegRNA), and one zinc finger nuclease (or nucleic acid encoding the zinc finger nuclease), wherein the zinc finger nuclease comprises a FokI cleavage domain or a modified FokI cleavage domain. The pegRNA directs the fusion protein to a specific chromosomal sequence, and the zinc finger nuclease is directed to another chromosomal sequence, wherein the fusion protein and the zinc finger nuclease dimerize such that the cleavage domain of the fusion protein and the cleavage domain of the zinc finger nuclease can introduce a double stranded break into the chromosomal sequence. See FIG. 1B. In embodiments in which the optional donor polynucleotide is not present, the resultant double-stranded breaks can be repaired by a non-homologous repair process such that deletions of at least one nucleotide, insertions of at least one nucleotide, substitutions of at least one nucleotide, or combinations thereof can occur during the repair of the break. In embodiments in which the optional donor polynucleotide is present, the donor sequence in the donor polynucleotide can be exchanged with or integrated into the chromosomal sequence during repair of the double-stranded break by either a homology-based repair process (e.g., in embodiments in which the donor sequence is flanked by upstream and downstream sequences having substantial sequence identity with upstream and downstream sequences, respectively, of the targeted sites in the chromosomal sequence) or a non-homologous repair process (e.g., in embodiments in which the donor sequence is flanked by compatible overhangs).

In still other embodiments in which the effector domain of the fusion protein is a transcriptional activation domain or a transcriptional repressor domain, the method can comprise introducing into the cell or embryo one fusion protein (or nucleic acid encoding one fusion protein) and one pegRNA (or DNA encoding one pegRNA). The pegRNA directs the fusion protein to a specific chromosomal sequence, wherein the transcriptional activation domain or a transcriptional repressor domain activates or represses expression, respectively, of the targeted chromosomal sequence. See FIG. 2A.

In alternate embodiments in which the effector domain of the fusion protein is an epigenetic modification domain, the method can comprise introducing into the cell or embryo one fusion protein (or nucleic acid encoding one fusion protein) and one pegRNA (or DNA encoding one pegRNA). The pegRNA directs the fusion protein to a specific chromosomal sequence, wherein the epigenetic modification domain modifies the structure of the targeted the chromosomal sequence. See FIG. 2B. Epigenetic modifications include acetylation, methylation of histone proteins and/or nucleotide methylation. In some instances, structural modification of the chromosomal sequence leads to changes in expression of the chromosomal sequence.

### (VI) Genetically Modified Cells and Animals

The present disclosure encompasses genetically modified cells, non-human embryos, and non-human animals comprising at least one chromosomal sequence that has been modified using an RNA-guided endonuclease-mediated or fusion protein-mediated process, for example, using the methods described herein. The disclosure provides cells comprising at least one DNA or RNA molecule encoding an RNA-guided endonuclease or fusion protein targeted to a chromosomal sequence of interest or a fusion protein, at least one pegRNA, and optionally one or more donor polynucleotide(s). The disclosure also provides non-human embryos comprising at least one DNA or RNA molecule encoding an RNA-guided endonuclease or fusion protein targeted to a chromosomal sequence of interest, at least one pegRNA, and optionally one or more donor polynucleotide(s).

The present disclosure provides genetically modified non-human animals, non-human embryos, or animal cells comprising at least one modified chromosomal sequence. The modified chromosomal sequence may be modified such that it is (1) inactivated, (2) has an altered expression or produces an altered protein product, or (3) comprises an integrated sequence. The chromosomal sequence is modified with an RNA guided endonuclease-mediated or fusion protein-mediated process, using the methods described herein.

As discussed, one aspect of the present disclosure provides a genetically modified animal in which at least one chromosomal sequence has been modified. In one embodiment, the genetically modified animal comprises at least one inactivated chromosomal sequence. The modified chromosomal sequence may be inactivated such that the sequence is not transcribed and/or a functional protein product is not produced. Thus, a genetically modified animal comprising an inactivated chromosomal sequence may be termed a “knock out” or a “conditional knock out.” The inactivated chromosomal sequence can include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted chromosomal sequence is inactivated and a functional protein is not produced. The inactivated chromosomal sequence comprises no exogenously introduced sequence. Also included herein are genetically modified animals in which two, three, four, five, six, seven, eight, nine, or ten or more chromosomal sequences are inactivated.

In another embodiment, the modified chromosomal sequence can be altered such that it codes for a variant protein product. For example, a genetically modified animal comprising a modified chromosomal sequence can comprise a targeted point mutation(s) or other modification such that an altered protein product is produced. In one embodiment, the chromosomal sequence can be modified such that at least one nucleotide is changed and the expressed protein comprises one changed amino acid residue (missense mutation). In another embodiment, the chromosomal sequence can be modified to comprise more than one missense mutation such that more than one amino acid is changed. Additionally, the chromosomal sequence can be modified to have a three nucleotide deletion or insertion such that the expressed protein comprises a single amino acid deletion or insertion. The altered or variant protein can have altered properties or activities compared to the wild type protein, such as altered substrate specificity, altered enzyme activity, altered kinetic rates, etc.

In another embodiment, the genetically modified animal can comprise at least one chromosomally integrated sequence. A genetically modified animal comprising an integrated sequence may be termed a “knock in” or a “conditional knock in.” The chromosomally integrated sequence can, for example, encode an orthologous protein, an endogenous protein, or combinations of both. In one embodiment, a sequence encoding an orthologous protein or an endogenous protein can be integrated into a chromosomal sequence encoding a protein such that the chromosomal sequence is inactivated, but the exogenous sequence is expressed. In such a case, the sequence encoding the orthologous protein or endogenous protein may be operably linked to a promoter control sequence. Alternatively, a sequence encoding an orthologous protein or an endogenous protein may be integrated into a chromosomal sequence without affecting expression of a chromosomal sequence. For example, a sequence encoding a protein can be integrated into a “safe harbor” locus, such as the Rosa26 locus, HPRT locus, or AAV locus. The present disclosure also encompasses genetically modified animals in which two, three, four, five, six, seven, eight, nine, or ten or more sequences, including sequences encoding protein(s), are integrated into the genome.

The chromosomally integrated sequence encoding a protein can encode the wild type form of a protein of interest or can encode a protein comprising at least one modification such that an altered version of the protein is produced. For example, a chromosomally integrated sequence encoding a protein related to a disease or disorder can comprise at least one modification such that the altered version of the protein produced causes or potentiates the associated disorder. Alternatively, the chromosomally integrated sequence encoding a protein related to a disease or disorder can comprise at least one modification such that the altered version of the protein protects against the development of the associated disorder.

In an additional embodiment, the genetically modified animal can be a “humanized” animal comprising at least one chromosomally integrated sequence encoding a functional human protein. The functional human protein can have no corresponding ortholog in the genetically modified animal. Alternatively, the wild type animal from which the genetically modified animal is derived may comprise an ortholog corresponding to the functional human protein. In this case, the orthologous sequence in the “humanized” animal is inactivated such that no functional protein is made and the “humanized” animal comprises at least one chromosomally integrated sequence encoding the human protein.

In yet another embodiment, the genetically modified animal can comprise at least one modified chromosomal sequence encoding a protein such that the expression pattern of the protein is altered. For example, regulatory regions controlling the expression of the protein, such as a promoter or a transcription factor binding site, can be altered such that the protein is over-produced, or the tissue-specific or temporal expression of the protein is altered, or a combination thereof. Alternatively, the expression pattern of the protein can be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomal sequence. The genetically modified animal comprising the lox-flanked chromosomal sequence can then be crossed with another genetically modified animal expressing Cre recombinase. Progeny animals comprising the lox-flanked chromosomal sequence and the Cre recombinase are then produced, and the lox-flanked chromosomal sequence is recombined, leading to deletion or inversion of the chromosomal sequence encoding the protein. Expression of Cre recombinase can be temporally and conditionally regulated to effect temporally and conditionally regulated recombination of the chromosomal sequence.

In any of these embodiments, the genetically modified animal disclosed herein can be heterozygous for the modified chromosomal sequence. Alternatively, the genetically modified animal can be homozygous for the modified chromosomal sequence.

The genetically modified animals disclosed herein can be crossbred to create animals comprising more than one modified chromosomal sequence or to create animals that are homozygous for one or more modified chromosomal sequences. For example, two animals comprising the same modified chromosomal sequence can be crossbred to create an animal homozygous for the modified chromosomal sequence. Alternatively, animals with different modified chromosomal sequences can be crossbred to create an animal comprising both modified chromosomal sequences.

For example, a first animal comprising an inactivated chromosomal sequence gene “x” can be crossed with a second animal comprising a chromosomally integrated sequence encoding a human gene “X” protein to give rise to “humanized” gene “X” offspring comprising both the inactivated gene “x” chromosomal sequence and the chromosomally integrated human gene “X” sequence. Also, a humanized gene “X” animal can be crossed with a humanized gene “Y” animal to create humanized gene X/gene Y offspring. Those of skill in the art will appreciate that many combinations are possible.

In other embodiments, an animal comprising a modified chromosomal sequence can be crossbred to combine the modified chromosomal sequence with other genetic backgrounds. By way of non-limiting example, other genetic backgrounds may include wild-type genetic backgrounds, genetic backgrounds with deletion mutations, genetic backgrounds with another targeted integration, an genetic backgrounds with non-targeted integrations.

The term “animal,” as used herein, refers to a non-human animal. The animal may be an embryo, a juvenile, or an adult. Suitable animals include vertebrates such as mammals, birds, reptiles, amphibians, shellfish, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. Alternatively, the animal may be an invertebrate such as an insect, a nematode, and the like. Non-limiting examples of insects include Drosophila and mosquitoes. An exemplary animal is a rat. Non-limiting examples of suitable rat strains include Dahl Salt-Sensitive, Fischer 344, Lewis, Long Evans Hooded, Sprague-Dawley, and Wistar. In one embodiment, the animal is not a genetically modified mouse. In each of the foregoing iterations of suitable animals for the invention, the animal does not include exogenously introduced, randomly integrated transposon sequences.

A further aspect of the present disclosure provides genetically modified cells or cell lines comprising at least one modified chromosomal sequence. The genetically modified cell or cell line can be derived from any of the genetically modified animals disclosed herein. Alternatively, the chromosomal sequence can be modified in a cell as described herein above (in the paragraphs describing chromosomal sequence modifications in animals) using the methods descried herein. The disclosure also encompasses a lysate of said cells or cell lines.

In general, the cells are eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells can be primary cells. In general, any primary cell that is sensitive to double strand breaks may be used. The cells may be of a variety of cell types, e.g., fibroblast, myoblast, T or B cell, macrophage, epithelial cell, and so forth.

When mammalian cell lines are used, the cell line can be any established cell line or a primary cell line that is not yet described. The cell line can be adherent or non-adherent, or the cell line can be grown under conditions that encourage adherent, non-adherent or organotypic growth using standard techniques known to individuals skilled in the art. Non-limiting examples of suitable mammalian cells and cell lines are provided herein in section (IV)(g). In still other embodiments, the cell can be a stem cell. Non-limiting examples of suitable stem cells are provided in section (IV)(g).

The present disclosure also provides a genetically modified non-human embryo comprising at least one modified chromosomal sequence. The chromosomal sequence can be modified in an embryo as described herein above (in the paragraphs describing chromosomal sequence modifications in animals) using the methods descried herein. In one embodiment, the embryo is a non-human fertilized one-cell stage embryo of the animal species of interest. Exemplary mammalian embryos, including one cell embryos, include without limit, mouse, rat, hamster, rodent, rabbit, feline, canine, ovine, porcine, bovine, equine, and primate embryos.

### EXEMPLIFICATION

### Cis-Acting Regulatory Elements Greatly Enhance the Efficiency of PE2 and PE3

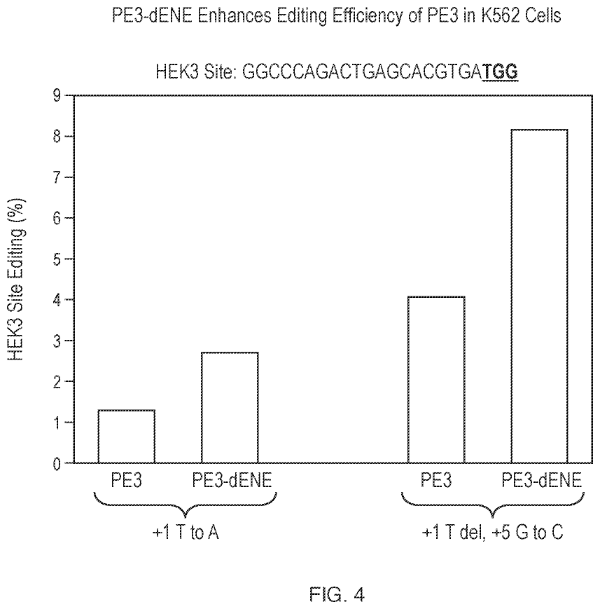

In the proof-of-concept experiment, the wild-type Double Elements for Nuclease Expression (dENE) from rice TWIFB1 was added immediately to the 3′-end of prime editor expression cassette after the stop codon and before the mRNA terminator, e.g., BGH as shown in FIG. 2, where dENE is reverse transcribed into RNA sequence that is included in prime editor mRNA. K562 cells were nucleofected with prime editor (PE2)-expressing construct with or without dENE sequence contained in the construct and together with pegRNA-expressing constructs targeting HEK3 site for different types of editing as indicated in FIG. 3 and FIG. 4. In case of PE3, one additional nicking guide RNA-expressing construct was added to the nucleofection mix. Cells were harvested three days post nucleofection for Next Generation Sequencing (NGS) analysis for prime editing efficiency. As shown, inclusion of a 3′-UTR dENE to prime editor (PE2)-expressing cassette greatly enhanced editing efficiency up to 8 fold on HEK3 target for the editing of +1 CTT insertion and +5 G deletion (FIG. 3), and up to 2 fold on HEK3 target for the editing of +1 T to A conversion, and the editing of +1 T deletion and +5 G-to-C conversion in case of PE3 (FIG. 4).

### Cis-Acting Regulatory Elements Enhance Prime Editing Efficiency in a Cell-Type Dependent Manner

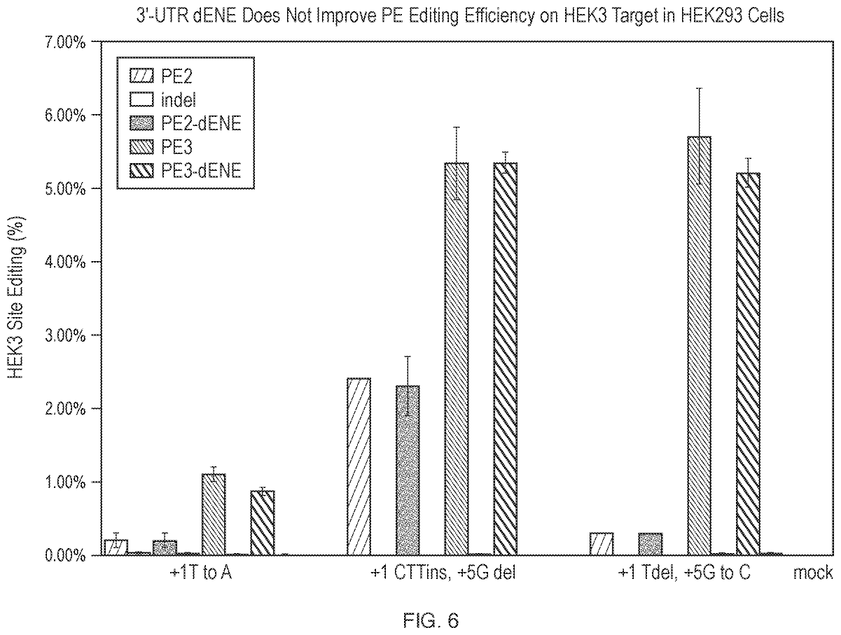

In two separate experiments, K562 or HEK293 cells were nucleofected with prime editor (PE2)-expressing construct with or without dENE sequence contained in the construct and together with pegRNA-expressing constructs targeting HEK3 site (GGCCCAGACTGAGCACGTGATGG [SEQ ID NO: 26], underlined bases indicates PAM sequence) for different types of editing as indicated in FIG. 5 and FIG. 6. In cases of PE3, one additional nicking guide RNA-expressing construct was added to the nucleofection mix. Cells were harvested three days post nucleofection for Next Generation Sequencing (NGS) analysis for prime editing efficiency. As shown in FIG. 5, inclusion of a 3′-UTR dENE to prime editor (PE2)-expressing cassette enhanced editing efficiency by approximately 50% on HEK3 target for the editing of +1 CTT insertion and +5 G deletion, and similarly approximately 50% on HEK3 target for the editing of +1 T to A conversion, +1 CTT insertion and +5 G deletion, and +1T deletion and +5G to C in cases of PE3 in K562 cells. Instead, inclusion of a 3′-UTR dENE to prime editor (PE2)-expressing cassette was not demonstrated to enhance editing efficiency on the same HEK3 target for the same types of editing and in cases of PE3 in HEK293 cells, as shown in FIG. 6. This indicates that 3′-UTR element dENE works to enhance prime editing efficiency in a cell type-dependent manner on the tested target in certain cell types.

We claim:

1) A synthetic nucleic acid composition comprising: i) a sequence encoding a CRISPR-Cas protein, ii) a sequence encoding a reverse transcriptase, and iii) a sequence encoding a cis-acting regulatory element.

2) The synthetic nucleic acid composition of claim 1, wherein said CRISPR-Cas protein is nCas9-H840A.

3) The synthetic nucleic acid composition of claim 1, wherein said reverse transcriptase is M-MLV-RT.

4) The synthetic nucleic acid composition of claim 1, wherein said cis-acting regulatory element is dENE or ENE.

5) The synthetic nucleic acid composition of claim 1, wherein said cis-acting regulatory element is sRSM1.

6) The synthetic nucleic acid composition of claim 1, wherein said nucleic acid is DNA.

7) The synthetic nucleic acid composition of claim 1, wherein said nucleic acid is RNA.

8) The synthetic nucleic acid composition of claim 1, wherein said composition further comprises an expression promotor.

9) The synthetic nucleic acid composition of claim 8, wherein said composition is in an expression vector.

10) The synthetic nucleic acid composition of claim 8, wherein said composition is incorporated into a transfection virus.

11) The synthetic nucleic acid composition of claim 1, wherein said cis-acting regulatory element is located after the stop codon of the CRISPR-Cas9 sequence and before an mRNA terminator.

12) The synthetic nucleic acid composition of claim 1, further comprising a prime editing guide RNA (pegRNA), wherein said pegRNA is derived from one of PE1, PE2 and PE2.

13) An amino acid sequence encoded by the synthetic nucleic acid composition of claim 1.

14) A method of modifying an endogenous DNA sequence, comprising:
a) providing: i) an operable expression vector comprising a synthetic nucleic acid composition comprising: 1) a sequence encoding a CRISPR-Cas type II system protein, 2) a sequence encoding a reverse transcriptase, and 3) a sequence comprising a cis-acting regulatory element; ii) a prime editing guide RNA (pegRNA) comprising a prime binding site (PBS); and iii) a cell comprising a target endogenous DNA sequence being at least 50% complementary to the PBS;
b) transfecting the cell comprising the endogenous DNA sequence of interest with the synthetic nucleic acid composition and pegRNA of the present invention; and
c) culturing said transfected cell such that the desired modification is made to the endogenous DNA sequence.

15) The method of claim 14, wherein said CRISPR-Cas type II system protein is a Cas9 protein.

16) The method of claim 14, wherein said endogenous DNA sequence is at least 75% complementary to the PBS.

17) The method of claim 14, wherein said endogenous DNA sequence is at least 90% complementary to the PBS.

18) The method of claim 14, wherein said endogenous DNA sequence is at least 95% complementary to the PBS.

19) The method of claim 14, wherein said endogenous DNA sequence is at least 98% complementary to the PBS.

20) The method of claim 14, wherein said endogenous DNA sequence is 100% complementary to the PBS.

21) The method of claim 14, wherein said CRISPR-Cas protein is nCas9-H840A.

22) The method of claim 14, wherein said reverse transcriptase is M-MLV-RT.

23) The method of claim 14, wherein said cis-acting regulatory element is Dene or ENE.

24) The method of claim 14, wherein said cis-acting regulatory element is sRSM1.

25) The method of claim 14, wherein said operable expression vector is DNA.

26) The method of claim 14, wherein said operable expression vector is RNA.

27) The synthetic nucleic acid composition of claim 14, wherein said composition is incorporated into a transfection virus.

28) The synthetic nucleic acid composition of claim 14, wherein said cis-acting regulatory element is located after the stop codon of the CRISPR-Cas9 sequence and before an mRNA terminator.

29) The method of any of claim 14, wherein said pegRNA is derived from one of PE1, PE2 and PE3.

30) The method of any of claim 14, wherein said CRISPR/Cas type II system protein is introduced into the cell encoded in an operable expression vector.
